(19) United States
(12) Patent Application Publication
(10) Pub. No.: US 2025/0082733 A1
(43) Pub. Date: Mar. 13, 2025

(54) m6A mRNA MODIFICATION IN CANCER TREATMENT

(71) Applicant: CITY OF HOPE, Duarte, CA (US)

(72) Inventors: Yanhong SHI, Arcadia, CA (US); Qi CUI, Duarte, CA (US)

(73) Assignee: CITY OF HOPE, Duarte, CA (US)

(21) Appl. No.: 18/814,226

(22) Filed: Aug. 23, 2024

Related U.S. Application Data
(63) Continuation of application No. 16/494,247, filed on Sep. 13, 2019, now abandoned.
(60) Provisional application No. 62/470,681, filed on Mar. 13, 2017.

(51) Int. Cl. A61K 38/45; A61K 31/196; A61K 31/245; A61K 31/713; A61K 48/00; A61P 35/00; C12N 9/10; C12N 15/113

(52) U.S. Cl. CPC A61K 38/45; A61K 31/196; A61K 31/245; A61K 31/713; A61P 35/00; C12N 9/1007; C12N 15/1137; C12Y 201/01062; A61K 48/00; C12N 2310/122; C12N 2310/14; C12N 2310/141; C12N 2310/531

(57) ABSTRACT

Disclosed herein are methods of treating cancer by increasing mRNA m6A methylation level and/or decreasing mRNA m6A demethylation in cancer stem cells. The methods entail administering an effective amount of one or more therapeutic agents to the subject. The therapeutic agents include an agent that induces overexpression of METTL3, an agent that induces overexpression of METTL14, an agent that inhibits FTO, an agent that inhibits ALKBH5, and an agent that inhibits TLX. Also disclosed are pharmaceutical compositions for treating cancer, which compositions include one or more such therapeutic agents.

### PRIORITY CLAIM

The present invention is a continuation of U.S. patent application Ser. No. 16/494,247, filed Sep. 13, 2019, which is a U.S. national phase application of International Patent Application No. PCT/US2018/022231, filed Mar. 13, 2018, which claims the benefit of U.S. Provisional Patent Application No. 62/470,681, filed Mar. 13, 2017, the content of which are all incorporated herein by reference in their entirety.

### STATEMENT OF GOVERNMENT INTEREST

The present invention was made with government support under Grant Nos. RB4-06277 and TRAN1-08525, awarded by California Institute for Regenerative Medicine. The Government has certain rights in the invention.

### SEQUENCE LISTING

This application contains a ST.26 compliant Sequence Listing, which was submitted in XML format via Patent Center, and is hereby incorporated by reference in its entirety. The XML copy, created on Nov. 12, 2024, is named 0544358167US02.xml and is 27,000 bytes in size.

### BACKGROUND

Glioblastoma is the most deadly primary brain tumor. Even with the combined surgical resection, radiation therapy, and chemotherapy, median survival of patients is less than 15 months after diagnosis (Stupp et al., 2009; Johnson and O'Neill, 2012). Lack of success in treating glioblastoma likely arises from tumor heterogeneity and the treatment resistance of glioblastoma stem cells (GSCs), a population of cancer stem cells with extraordinary capacity to promote tumor growth, invasion, and display increased resistance to radiotherapy and chemotherapy (Singh et al., 2004; Bao et al., 2006; Godlewski et al., 2010). The presence of these cancer stem cells renders glioblastoma treatment-resistant and recurring (Sundar et al., 2014). Therefore new cancer therapies, including glioblastoma therapies, that target these treatment-resistant cancer stem cells are urgently needed (Godlewski et al., 2010; Allegra et al., 2014).

### SUMMARY

In one aspect, the disclosure provided herein relates to a method of treating cancer in a subject by promoting m6A modification and/or decreasing m6A demethylation. The method entails administering a therapeutic effective amount of one or more therapeutic agents to the subject to increase the expression of methyltransferase-like 3 (METTL3) or methyltransferase-like 14 (METTL14), or to inhibit the expression of fat mass and obesity-associated protein (FTO) gene, alkylation repair homologue protein 5 (ALKBH5) gene, or nuclear receptor TLX gene. Alternatively, the method entails knocking out the FTO gene, the ALKBH5 gene, or the TLX gene, e.g., by CRISPR-Cas9 or other known methods in the art. In some embodiments, the cancer is an m⁶A related cancer, such as glioblastoma, leukemia (e.g., acute myeloid leukemia), stomach cancer, prostate cancer, colorectal cancer, endometrial cancer, breast cancer, pancreatic cancer, kidney cancer, mesothelioma, sarcoma, etc.

In another aspect, the disclosure provided herein relates to a pharmaceutical composition comprising a therapeutically effective amount of one or more therapeutic agents that increase the expression or activity of METTL3 or METTL14, or that inhibit the expression or activity of FTO, ALKBH5, or TLX. The pharmaceutical composition may comprise one or more additional ingredients, such as a pharmaceutically acceptable excipient, carrier, diluent, surfactant, diluent, preservative, etc. The pharmaceutical composition can be formulated into any dosage form suitable for a particular administration route, such as parenteral injection, oral administration, intracranial injection, etc. The pharmaceutical composition can be in liquid, semi-solid (e.g., gel), or solid formulation. In some embodiments, the pharmaceutical composition includes a small molecule that inhibits FTO, ALKBH5, or TLX, or an anti-FTO, anti-ALKBH5, or anti-TLX antibody. In some embodiments, the small molecule FTO inhibitor is a meclofenamic acid (MA) or a derivative thereof, such as MA2. Additionally, the pharmaceutical composition can also include small RNAs such as miRNA, siRNAs or shRNAs for inhibiting FTO, ALKBH5, or TLX.

### BRIEF DESCRIPTION OF THE DRAWINGS

FIGS. 1a-1d show that differentiation of GSCs induced elevated levels of m⁶A RNA modification. 1a. A list of GSC lines used in this study. The characterization of these GSCs, including glioblastoma (GBM) subtype, marker (TLX and nestin) expression, multipotency and tumor formation capacity, is summarized in the table. 1b. Differentiation of GSCs into Tuj1-positive neurons (red) and GFAP-positive astrocytes (green) by treating cells with FBS together with retinoic acid. Scale bar: 25 μm. 1c. RNA dot blot analysis of m⁶A levels in proliferating (P) GSCs and differentiated (D) cells. 1d. Quantification of m⁶A level measured by RNA dot blot shown in panel 1c. N=4. ***p<0.001 by Student's t-test. Error bars are s.d. of the mean.

FIGS. 2a-2d show that knocking down METTL3 expression promoted the growth and self-renewal of GSCs. 2a. RT-PCR analysis of METTL3 expression in GSCs transduced with lentivirus expressing control shRNA (shC) or METTL3 shRNAs (shMETTL3-1, shMETTL3-2). N=3. See also FIG. 3. 2b to 2d. Cell growth (2b), sphere formation (2c), and limiting dilution assay (LDA) (2d) of GSCs transduced with lentivirus expressing control shRNA or METTL3 shRNAs. Sphere formation assay and LDA were used to evaluate the self-renewal capacity of GSCs. N=4 for 2b. N=6 for 2c. N=20 for 2d. *p<0.05, **p<0.01, ***p<0.001 by Student's t-test. Error bars are s.d. of the mean.

FIGS. 3a-3i show modulation of m⁶A mRNA modification by manipulating the m⁶A methylation machinery in GSCs. 3a, 3b. Western blot analysis of METTL3knockdown in Flag-METTL3 (Flag-M3)-expressing HEK293T (3a) and PBT003 (3b) cells. 3c, 3d. Western blot analysis of METTL14 knockdown in HEK293T (3c) and PBT707 (3d) cells. 3e, 3f. mRNA dot blot analysis of m⁶A levels in METTL3 or METTL14 knockdown PBT003 cells. shC: control shRNA; shMETTL3-1 and shMETTL3-2: shRNAs for METTL3; shMETTL14-1 and shMETTL14-2: shRNAs for METTL14. 3g. mRNA dot blot analysis of m⁶A levels in PBT007 cells transduced with METTL3-expressing virus or control virus. 3h. mRNA dot blot analysis of m⁶A levels in PBT007 cells treated with vehicle control or MA2. N=3. ***p<0.001 by Student's t-test. Error bars are s.e. of the mean. 3i. RT-PCR analysis of CD44 expression in PBT003 cells treated with control shRNA (shC), METTL3 shRNA 1 (shMETTL3-1), METTL14 shRNA 1 (shMETTL14-1) or METTL3 overexpression (OE). N=3. **p<0.01, ***p<0.001 by Student's t-test. Error bars are s.d. of the mean.

FIGS. 4a-4d show that knocking down METTL14 expression enhanced the growth and self-renewal of GSCs. 4a. RT-PCR analysis of METTL14 expression in GSCs transduced with lentivirus expressing control shRNA (shC) or METTL14 shRNAs (shMETTL14-1, shMETTL14-2). N=3. See also FIGS. 3. 4b to 4d. Cell growth (4b), sphere formation (4c), and LDA (4d) analyses of GSCs transduced with lentivirus expressing control shRNA or METTL14 shRNAs. N=4 for 4b. N=6 for 4c. N=20 for 4d. *p<0.05, **p<0.01, ***p<0.001 by Student's t-test. Error bars are s.d. of the mean.

FIGS. 5a-5c show that overexpressing METTL3 inhibited the growth and self-renewal of GSCs. 5a. RT-PCR analysis showing overexpression of METTL3 in GSCs. 5b, 5c. Cell growth (5b) and sphere formation (5c) analyses of GSCs transduced with METTL3-expressing virus or control virus. N=4 for 5b. N=6 for 5c. *p<0.05, **p<0.01, ***p<0.001 by Student's t-test. Error bars are s.d. of the mean.

FIGS. 6a-6e show that overexpressing the wild type, but not catalytically inactive METTL3 inhibited the growth and self-renewal of GSCs. 6a. RT-PCR analysis showing overexpression of the wild type (WT) or the catalytic mutant (Mut) METTL3 in GSCs (PBT003, PBT707, and PBT726 cells). 6b-6d. Cell growth (6b), sphere formation (6c), and LDA (6d) analyses of GSCs (PBT003, PBT707, and PBT726 cells) transduced with the control virus (C), the WT METTL3 (M3) or the catalytic mutant METTL3 (M3-mut)-expressing virus. 6e. Sphere formation assay of GSCs transduced with lentivirus expressing METTL3 shRNA (shM3) alone or together with the WT METTL3 (M3) or the catalytic mutant METTL3 (M3-mut). N=4 for 6b, 6c, 6e. N=20 for 6d. *p<0.05, **p<0.01, ***p<0.001 by Student's t-test. Error bars are s.d. of the mean.

FIGS. 7a-7e show that knocking down METTL3 and/or METTL14 expression promoted the tumorigenicity of GSCs. 7a. Schematic of the experimental design, including GSC transplantation and xenogen imaging of xenografted tumors. 7b. Xenogen images of brain tumors in NSG mice transplanted with PBT707 cells that were transduced with control shRNA (shC), METTL3 shRNA (shMETTL3) or METTL14 shRNA (shMETTL14). The scale bar for bioluminescence intensity is shown on the right. 7c, 7d. Quantification of the bioluminescence intensity of tumors at 8 weeks (7c) and 10 weeks (7d) after tumor transplantation. *p<0.05 and **p<0.01 by Student's t-test. Error bars are s.d. of the mean. 7e. The survival curves of NSG mice transplanted with PBT707 cells transduced with control shRNA (shC), METTL3 shRNA (shMETTL3) or METTL14 shRNA (shMETTL14). The X axis represents days after GSC transplantation. N=7, log-rank test.

FIGS. 8a-8d show that knocking down METTL3 expression enhanced the tumorigenicity of GSCs in PBT003 cells. 8a. Schematic of the experimental design, including GSC transplantation and xenogen imaging of xenografted tumors. 8b. Xenogen images of brain tumors in NSG mice transplanted with PBT003 cells that were transduced with control shRNA (shC) or METTL3 shRNA (shMETTL3). The scale bar for bioluminescence intensity is shown on the right. 8c. Quantification of the bioluminescence intensity of tumors. *p<0.05 by Student's t-test. Error bars are s.d. of the mean. 8d. The survival curves of NSG mice transplanted with PBT003 cells transduced with control shRNA (shC) or METTL3 shRNA (shMETTL3). The X axis represents days after GSC transplantation. N=7, log-rank test.

FIGS. 9a-9d show that knocking down METTL3 and METTL14 expression promoted the tumorigenicity of GSCs in PBT003 cells. 9a. Schematic of the experimental design, including GSC transplantation and xenogen imaging of xenografted tumors. 9b. Xenogen images of brain tumors in NSG mice transplanted with PBT003 cells that were transduced with control shRNA (shC), METTL14 shRNA (shM14), or the combination of METTL14 shRNA and METTL3 shRNA (shM3+shM14). The scale bar for bioluminescence intensity is shown on the right. 9c. Quantification of the bioluminescence intensity of tumors in NSG mice transplanted with PBT003 cells that were transduced with shC, shM14, or shM3+shM14. *p<0.05, **p<0.01 by Student's t-test. Error bars are s.d. of the mean. 9d. The survival curves of NSG mice transplanted with PBT003 cells transduced with shC or shM3+shM14. The X axis represents days after GSC transplantation. N=5, log-rank test.

FIGS. 10a-10c show that knocking down METTL3 or METTL14 expression increased the tumorigenicity of GSCs in PBT726 cells. 10a. Schematic of the experimental design, including GSC transplantation and xenogen imaging of xenografted tumors. 10b. Xenogen images of brain tumors in NSG mice transplanted with PBT726 cells that were transduced with control shRNA (shC), METTL3 shRNA (shMETTL3) or METTL14 shRNA (shMETTL14). The scale bar for bioluminescence intensity is shown on the right. 10c. Quantification of the bioluminescence intensity of tumors in NSG mice transplanted with PBT726 cells that were transduced with control shRNA (shC), METTL3 shRNA (shM3) or METTL14 shRNA (shM14). N=8, *p<0.05, **p<0.01 by Student's t-test. Error bars are s.d. of the mean.

FIGS. 11a-11g show that treatment with the FTO inhibitor MA2 reduced GSC-initiated tumor growth. 11a. Cell growth analyses of GSCs treated with the FTO inhibitor MA2. N=4. 11b. LDA analysis of GSCs treated with MA2 or vehicle control. N=20. 11c. Sphere formation analysis of GSCs treated with control shRNA (shC), METTL3 or METTL14 shRNA (shM3 or shM14) expressing virus alone or together with MA2. *p<0.05, **p<0.01, and ***p<0.001 by Student's t-test. Error bars are s.d. of the mean. 11d. Schematic of the experimental design, including GSC transplantation, MA2 treatment and xenogen imaging of tumors derived from grafted GSCs. The transplanted mice were treated with the FTO inhibitor MA2 or vehicle control. 11e. Xenogen images of brain tumors in GSC-grafted NSG mice treated with vehicle control (C) or MA2. The scale bar for bioluminescence intensity is shown on the right. 11f. Quantification of the bioluminescence intensity of tumors. N=10. *p<0.05 by Student's t-test. Error bars are s.d. of the mean. 11g. The survival curves of GSC-grafted NSG mice treated with MA2 or vehicle control. The X axis represents days after the first MA2 treatment. N=8, log-rank test.

FIGS. 12a-12b show that the FTO inhibitor MA2 suppressed the growth and self-renewal of GSCs. 12a. Cell growth analyses of neural stem cells (NSC 006), astrocytes, and Hela cells treated with the FTO inhibitor MA2. NSC006, astrocytes and HeLa cells were included as controls for GSCs shown in FIG. 11a. N=4. 12b. LDA analysis of GSCs treated with MA2 or vehicle control. N=20. ***p<0.001 by Student's t-test. Error bars are s.d. of the mean.

FIGS. 13a-13k show that METTL3 or METTL14 KD induced mRNA expression and m⁶A methylation level change in GSCs. 13a. Heatmap showing mRNA expression changes in PBT003 cells with METTL3 or METTL14 KD. shC: control shRNA; shM3: shRNA for METTL3; shM14-1 and shM14-2: shRNAs for METTL14. 13b, 13c. RT-PCR of ADAM19 (ADAM) (13b) and EPHA3 (13c) expression in PBT003 cells with METTL3 or METTL14 KD, METTL3 overexpression (OE), or MA2 treatment. N=3. 13d. GO analysis of genes with expression change upon METTL3 or METTL14 KD in PBT003 cells. 13e, 13f. The m⁶A motif (13e) and peak distribution (13f) in GSCs. 13g. Change of the m⁶A methylation level in ADAM19 mRNA in PBT003 cells with METTL14 KD. The mRNA input in shC and shM14-1 cells was included at the top panels. The mRNA pulled down by immunoprecipitation with an m⁶A antibody (m⁶A IP) was included at the bottom panels. 13h, 13i. Cell growth (13h) and sphere formation (13i) analyses of PBT003 cells transduced with lentivirus expressing shC or ADAM19 shRNAs (shADAM-1, shADAM-2). N=4. 13j, 13k. Sphere formation assay of PBT003 cells transduced with lentivirus expressing METTL3 shRNA (13j) or METTL14 shRNA (13k) alone or together with ADAM19 shRNA (shADAM). N=4. *p<0.05, **p<0.01, ***p<0.001 by Student's t-test. Error bars are s.d. of the mean.

FIGS. 14a-14n show that METTL3 or METTL 14 knockdown induced mRNA expression and m⁶A methylation level changes in GSCs. 14a. RT-PCR analysis of KLF4 expression in PBT003 cells treated with control shRNA (shC), METTL3 shRNA (shM3), METTL14 shRNA (shM14), or METTL3 overexpression (OE). N=3. *p<0.05 by Student's t-test. Error bars are s.d. of the mean. 14b. GO analysis of transcripts with m⁶A peaks in GSCs. 14c, 14i. RT-PCR analysis of ADAM19 (ADAM) expression in GSCs transduced with lentivirus expressing control shRNA (shC) or ADAM19 shRNAs (shADAM-1, shADAM-2). N=3. 14d-14f, 14j-14l. Cell growth (14d, 14j), sphere formation (14e, 14k), and LDA (14f, 14l) analyses of GSCs (PBT707 and PBT726 cells) transduced with lentivirus expressing control shRNA (shC) or ADAM19 shRNAs (shADAM-1, shADAM-2). N=4 for 14d, 14e, 14j, 14k. N=20 for 14f, 141. 14g-14h & 14m-14n. Sphere formation analyses of PBT707 (14g, 14h) and PBT726 (14m, 14n) cells transduced with lentivirus expressing METTL3 shRNA (shM3) or METTL14 shRNA (shM14) alone or together with ADAM19 shRNA (shADAM). N=4. *p<0.05, **p<0.01, ***p<0.001 by Student's t-test. Error bars are s.d. of the mean.

FIGS. 15a-15b show that elevated METTL3 (15a) or METTL14 (15b) was associated with better outcome of glioma patient survival.

FIGS. 16a-16f show that the nuclear receptor TLX was essential for GSC self-renewal and tumorigenecity. 16a. Schematic of the experimental design, including GSC transplantation, shRNA viral vector injection, and xenogen imaging of xenografted tumors. 16b. RT-PCR analysis showing TLX knockdown in vivo. N=3, **p<0.01 by Student's t-test. Error bars are s.e. of the mean. 16c. Xenogen images of brain tumors in NSG mice treated with virus expressing scrambled control (SC) or TLX shRNA (shTLX). The scale for bioluminescence intensity is shown on the right. 16d. Quantification of the bioluminescence intensity of tumors treated with scrambled control (SC) or TLX shRNA (shTLX) in the brains of engrafted NSG mice. N=6, **p<0.01 by Student's t-test. Error bars are s.e. of the mean. 16e. Survival curves of PBT003-engrafted NSG mice treated with virus expressing either scrambled control (SC) or TLX shRNA (shTLX). X axis represents days after viral injection. N=10 for each treatment group. p<0.05 by log-rank test. 16f. H&E staining of brain tumor tissues derived from transplanted PBT003 cells in NSG mice treated with scrambled control (SC) or TLX shRNA (shTLX). Scale bar: 1 mm. Xenogen images of NSG mice survived over 200 days after treatment with virus expressing TLX shRNA (shTLX). H&E staining showing typical tumor infiltration characteristics of glioblastoma. Scale bar: 50 μm.

FIGS. 17a-17d show that knockout or knockdown of TLX elevated m6A RNA modification. 17a. RNA dot blot analysis of m⁶A levels in TLX wild type mouse brain cells (WT) and TLX knockout mouse brain cells (TLX−/−). 17b. Quantification of m⁶A level measured by RNA dot blot shown in panel 17a. N=4. ***p<0.001 by Student's t-test. Error bars are s.d. of the mean. 17c. RNA dot blot analysis of m⁶A levels in GSCs treated with control shRNA (SC) and GSCs treated with TLX shRNA (shTLX). 17d. Quantification of m⁶A level measured by RNA dot blot shown in panel 17c. N=4. ***p<0.001 by Student's t-test. Error bars are s.d. of the mean.

FIG. 18 shows that knockdown of METTL3 reversed TLX knockdown-induced growth inhibition in GSCs. Cell growth and sphere formation analyses of PBT003, PBT707, PBT726, and PBT111 cells transduced with lentivirus expressing dox-inducible shTLX alone or together with dox-inducible shMETTL3 (shTLX or shMETTL3+shTLX). N=4 for cell growth analyses and N=6 for sphere formation analyses, error bars are s.d. of the mean. **p<0.01, ***p<0.001 by Student's t-test.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

More than 100 RNA modifications have been reported, including modifications within mRNAs (Machnicka et al., 2013), among which, N⁶-methyladenosine (m⁶A) modification is the most prevalent internal modification in eukaryotic mRNAs (Wei et al., 1975). Although discovered in the 1970s (Desrosiers et al., 1974; Wei and Moss, 1974; Dubin and Taylor, 1975; Perry et al., 1975), the physiological significance of m⁶A modification in mRNA has only been appreciated until recent years because of breakthrough findings of two mammalian RNA demethylases, the fat mass and obesity-associated protein (FTO) and alkylation repair homologue protein 5 (ALKBH5), which demonstrated that m⁶A methylation is a dynamic and reversible modification (Jia et al., 2011; Zheng et al., 2013). Transcriptome-wide m⁶A profiling further showed that m⁶A modification is presented in thousands of RNA transcripts with unique distribution patterns (Dominissini et al., 2012; Meyer et al., 2012).

The formation of m⁶A modification is catalyzed by a methyltransferase complex that contains methyltransferase-like 3 (METTL3), methyltransferase-like 14 (METTL14), and Wilm's tumor 1-associating protein (WTAP) in mammalian cells (Bokar et al., 1994; Liu et al., 2014; Ping et al., 2014; Wang et al., 2014). Knockdown (KD) of either METTL3 or METTL14 induces a substantial decrease in m⁶A level in mRNA (Liu et al., 2014; Wang et al., 2014). While m⁶A methyltransferases and demethylases act as its writers and erasers, respectively, m⁶A readers selectively bind to m⁶A-containing RNA to mediate downstream effects (Yue et al., 2015).

The roles of RNA modifications in biological processes have just begun to be appreciated. RNA modifications have been implicated in embryonic stem cell maintenance and differentiation (Batista et al., 2014; Wang et al., 2014; Geula et al., 2015), circadian rhythm modification (Fustin et al., 2013), heat shock response (Zhou et al., 2015), meiotic progression (Schwartz et al., 2013), and neuronal function (Lemkine et al., 2005). However, the function of the majority of RNA modifications found in mRNAs remains unknown. Specifically, the functional roles of m⁶A methylation in cancer initiation and progression remain to be determined. The identification of the writers, readers and erasers of m⁶A modification and the development of the m⁶A-seq technology set the foundation for the field to define the roles of m⁶A mRNA modification in cancer biology.

As demonstrated in the working examples, knockdown of METTL3 or METTL14 expression dramatically increased GSC growth and self-renewal. In contrast, overexpression of METTL3 and/or METTL14, or treatment with MA2, a chemical inhibitor of the RNA demethylase FTO, inhibited GSC growth and self-renewal considerably. By transplanting METTL3 shRNA or METTL14 shRNA-transduced GSCs into immunodeficient NOD SCID Gamma (NSG) mice, it was shown that knockdown of METTL3 or METTL14 expression led to substantial increase of GSC-initiated tumor progression in transplanted mouse brains. Furthermore, treatment with MA2, a chemical inhibitor of FTO, dramatically suppressed GSC-induced tumorigenesis and prolonged the lifespan in GSC-grafted animals.

As demonstrated in this disclosure, controlling mRNA m⁶A level is critical for maintaining GSC growth, self-renewal, and tumor development. Knockdown of METTL3 or METTL14 expression reduced mRNA m⁶A level, enhanced the growth and self-renewal of GSCs in vitro, and promoted the ability of GSCs to form brain tumors in vivo. In contrast, overexpression of METTL3 or treatment with the FTO inhibitor MA2, increased mRNA m⁶A level in GSCs and suppressed GSC growth. Moreover, treatment of GSCs with the FTO inhibitor MA2 suppressed GSC-initiated tumorigenesis and prolonged the lifespan of GSC-engrafted mice. The finding that the FTO inhibitor MA2 suppresses GSC-initiated brain tumor development suggests that m⁶A methylation could be a promising target for cancer therapy, e.g., anti-glioblastoma therapy.

This disclosure relates to mRNA m⁶A modification in regulating GSC self-renewal and tumorigenesis. Study of mRNA modification is a nascent field as yet, and the significance of this epigenetic mark in controlling cell growth and differentiation is just beginning to be appreciated. Although m⁶A is most abundant in the brain (Meyer et al., 2012), no study on the role of m6A modification in either brain development or brain disorders has been reported yet, except recent studies demonstrating a role of m6A in Drosophila neuronal function (Haussmann et al., 2016; Lence et al., 2016). Moreover, the role of m⁶A in cancer is only starting to be revealed (Zhang et al., 2016; Li et al., 2017). In some embodiments, disclosed herein is a correlation between mRNA m⁶A methylation and glioblastoma tumorigenesis, which represents an important step towards developing novel therapeutic strategies to treat cancer, such as glioblastoma by targeting m6A modification, its upstream regulators or downstream targets in GSCs.

RNA epigenetics has become a fast-moving research field in biology and holds great promise for future therapeutic development for human diseases. The m⁶A modification, installed by a methyltransferase complex consisting of METTL3 and METTL14, is one of the most common and abundant modifications on mRNAs in eukaryotes. The evidence is clear that m6A methylation is more than a mere “decoration” of the mRNA. The reversible nature of m6A methylation strongly suggests a regulatory role for this RNA modification (Sibbritt et al., 2013). Such a role could be important during dynamic cell growth and differentiation process. Indeed, a role for m⁶A modification in controlling embryonic stem cell pluripotency and differentiation has been reported (Batista et al., 2014; Wang et al., 2014; Chen et al., 2015; Geula et al., 2015). Although components of the m⁶A methylation machinery have been linked to cancer (Linnebacher et al., 2010; Kaklamani et al., 2011; Pierce et al., 2011; Machiela et al., 2012; Long et al., 2013; Lin et al., 2016; Zhang et al., 2016), whether the effect is dependent on m⁶A modification remains to be clarified. A recent study demonstrated that METTL3 enhances translation in cancer cells independently of m⁶A modification (Lin et al., 2016). On the other hand, elevated levels of S-adenosyl methionine (SAM) donor of the methyl group in m⁶A methylation process has been shown to suppress cell growth in cancer (Pascale et al., 2002; Pakneshan et al., 2004; Guruswamy et al., 2008; Lu et al., 2009; Zhao et al., 2010). However, whether the growth-inhibitory effect of increased levels of SAM is caused by elevated levels of m⁶A modification remains unknown. A direct causative link between mRNA m⁶A methylation and tumorigenesis remains to be established (Sibbritt et al., 2013). Disclosed herein is the biological significance of m⁶A modification in glioblastoma biology by defining the role of m⁶A modification in GSC self-renewal and tumorigenesis via targeting multiple components of the m⁶A regulatory machinery, including METTL3, METTL14 and FTO.

This disclosure identified roles of m⁶A modification in glioblastoma, the most aggressive and invariably lethal brain tumor. GSCs which are implicated in the initiation and development of glioblastoma were studied. The results demonstrate that modulation of mRNA m⁶A level impacts multiple aspects of GSCs, including GSC growth, self-renewal, and tumorigenesis, suggesting that mRNA m⁶A modification may serve as promising targets for GSCs. Furthermore, m⁶A-seq analysis was performed in GSCs with knockdown of METTL3 or METTL14. This transcriptome-wide analysis revealed genes and pathways that are impacted by mRNA m⁶A modification in GSCs, which could also serve as potential molecular targets to inhibit GSC tumorigenesis for the treatment of glioblastoma. Additionally, as demonstrated herein, nuclear receptor TLX is essential for GSC self-renewal and tumorigenicity and can be an upstream regulator of m⁶A RNA modification in GSCs. M6A RNA modification is increased by TLX knockout or knockdown. Moreover, the TLX knockdown-induced growth inhibition in GSCs is reversed by knockdown of METTL3.

In one aspect, disclosed herein is a method for treating a subject suffering from a cancer. The method includes increasing mRNA m⁶A methylation level in the cancer stem cells. In some embodiments, the mRNA m⁶A methylation level is increased by overexpressing METTL3, overexpressing METTL14, inhibiting FTO, ALKBH5 or TLX, or a combination thereof. For example, the promoters of METTL3 or METTL14 can be activated and/or CRISPRa can be used to promote overexpression of METTL3 or METTL14. Moreover, administration of METTL3 mRNA, protein or a functional fragment thereof and/or administration of METTL14 mRNA, protein or a functional fragment thereof, and/or administration of small molecules that activates the activity of METT3 or METTL14 can also be used as a therapy. In some embodiments, the method entails administering one or more doses of a therapeutically effective amount of an FTO inhibitor, an ALKBH5 inhibitor, and/or a TLX inhibitor to the subject. FTO inhibitors are known in the art, such as small molecule FTO inhibitors and FTO antibodies or fragments thereof. Various small molecule FTO inhibitors have been developed and known in the art, including but not limited to MA, MA2 or other MA derivatives, 4-chloro-6-(6′-chloro-7′-hydroxy-2′,4′,4′-trimethyl-chroman-2′-yl)benzene-1,3-diol (CHTB), etc. FTO antibodies or immunogenic fragments of the antibodies, can be used for treating m⁶A methylation-related cancer. It is known in the art that polyclonal antibodies, monoclonal antibodies, human antibodies, and humanized antibodies can be used in therapy. Moreover, antibody fragments, including but not limited to, Fab fragments, Fab′ fragments, Fc fragments, and scFV fragments, can also be used as therapeutic agents. Alternatively, FTO gene can be knocked out by a known technique, for example, gene mutation, gene deletion, recombination, small RNAs (miRNA, siRNAs, shRNAs) or CRISPR/Cas9. Likewise, ALKBH5 small molecule inhibitors, antibodies, or small RNAs (miRNAs, siRNAs, shRNAs) or known gene knockout methods such as CRISPA/Cas9, can also be used in therapy. Furthermore, small RNAs (miRNAs, siRNAs, shRNAs) that inhibit TLX or known gene knockout methods such as CRISPA/Cas9 can be used to knock out or knock down TLX.

The term “subject” or “patient” as used herein refers to a subject who is suffering from a cancer condition, e.g., an m⁶A related cancer, such as glioblastoma, leukemia (e.g., acute myeloid leukemia), stomach cancer, prostate cancer, colorectal cancer, endometrial cancer, breast cancer, pancreatic cancer, kidney cancer, mesothelioma, sarcoma, etc. The subject or patient can be an animal, a mammal, or a human.

The terms “treat,” “treating,” and “treatment” as used herein with regard to a cancer condition refer to alleviating the condition partially or entirely, or eliminating, reducing, or slowing the development of one or more symptoms associated with the condition. In some embodiments, the term “treat,” “treating,” or “treatment” means that one or more symptoms of the cancer condition or complications are alleviated in a subject receiving the treatment as disclosed herein comparing to a subject who does not receive such treatment.

The phrase “an effective amount” or “a therapeutically effective amount” as used herein refers to an amount of a therapeutic agent that produces a desired therapeutic effect. For example, an effective amount of an FTO inhibitor may refer to that amount that treats cancer, e.g., glioblastoma. The precise effective amount is an amount of the therapeutic agent that will yield the most effective results in terms of efficacy in a given subject. This amount will vary depending upon a variety of factors, including but not limited to the characteristics of the therapeutic agent (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a compound and adjusting the dosage accordingly. For additional guidance, see Remington: The Science and Practice of Pharmacy (Gennaro ed. 20^{th} edition, Williams & Wilkins PA, USA) (2000).

According to some embodiments, the therapeutic agents administered to a subject that induce overexpression of METTL3 or METTL14 or that inhibit FTO may be part of a pharmaceutical composition. Such a pharmaceutical composition may include one or more of an agent that induces overexpression of METTL3, an agent that induces overexpression of METTL14, and an agent that inhibits FTO, ALKBH5, and/or TLX, and optionally a pharmaceutically acceptable carrier.

A “pharmaceutically acceptable carrier” as used herein refers to a pharmaceutically acceptable material, composition, or vehicle that is involved in carrying or transporting an agent or cell of interest from one tissue, organ, or portion of the body to another tissue, organ, or portion of the body. Such a carrier may comprise, for example, a liquid, solid, or semi-solid filler, solvent, surfactant, diluent, excipient, adjuvant, binder, buffer, dissolution aid, solvent, encapsulating material, sequestering agent, dispersing agent, preservative, lubricant, disintegrant, thickener, emulsifier, antimicrobial agent, antioxidant, stabilizing agent, coloring agent, or some combination thereof. Each component of the carrier is “pharmaceutically acceptable” in that it must be compatible with the other ingredients of the composition and must be suitable for contact with any tissue, organ, or portion of the body that it may encounter, meaning that it must not carry a risk of toxicity, irritation, allergic response, immunogenicity, or any other complication that excessively outweighs its therapeutic benefits.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) natural polymers such as gelatin, collagen, fibrin, fibrinogen, laminin, decorin, hyaluronan, alginate and chitosan; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as trimethylene carbonate, ethyl oleate and ethyl laurate; (13) agar; (14) buffering agents, such as magnesium hydroxide and aluminum hydroxide; (15) alginic acid (or alginate); (16) pyrogen-free water; (17) isotonic saline; (18) Ringer's solution; (19) alcohol, such as ethyl alcohol and propane alcohol; (20) phosphate buffer solutions; (21) thermoplastics, such as polylactic acid, polyglycolic acid, (22) polyesters, such as polycaprolactone; (23) self-assembling peptides; and (24) other non-toxic compatible substances employed in pharmaceutical formulations such as acetone.

The pharmaceutical compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

In one embodiment, the pharmaceutically acceptable carrier is an aqueous carrier, e.g. buffered saline and the like. In certain embodiments, the pharmaceutically acceptable carrier is a polar solvent, e.g. acetone and alcohol.

In some embodiments, one or more therapeutic agents disclosed above are administered to the subject simultaneously. In some embodiments, one or more therapeutic agents disclosed above are administered to the subject sequentially. The term “simultaneously” as used herein with regards to administration means that one therapeutic agent is administered to the subject at the same time or nearly at the same time of administering another therapeutic agent. For example, two or more therapeutic agents are considered to be administered “simultaneously” if they are administered via a single combined administration, two or more administrations occurring at the same time, or two or more administrations occurring in succession without extended intervals in between.

When multiple doses and/or one or more therapeutic agents are administered, it is within the purview of one of ordinary skill in the art to adjust the administration schedule to optimize the therapeutic effect. In some embodiments, one or more doses and/or one or more therapeutic agents can be administered subsequently after the administration of the first dose or the first therapeutic agent, e.g., within one month of administration of the first dose or first therapeutic agent. For example, the subsequent doses of the therapeutic agent can be administered in one-week intervals or in two-week intervals for an extended period of time, e.g., up to one year. In some embodiments, one or more doses and/or one or more therapeutic agents are administered on a daily basis, every other day, every other two days, or on a weekly basis.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### EXAMPLES

### Experimental Procedures

Cell culture: GSCs derived from patients that were newly diagnosed as grade IV glioblastoma were maintained in sphere cultures as previously described (Cui et al., 2016). Briefly, GSCs were cultured in DMEM-F12 medium (Omega Scientific) supplemented with 1 X B27 (Invitrogen), 2 mM L-glutamine (Media Tech), 27.4 mM HEPES (Fisher) and growth factors including 20 ng ml⁻¹ EGF (PeproTech), 20 ng ml⁻¹ FGF (PeproTech) and 5 μg ml⁻¹ heparin (Sigma). All cultures were confirmed for no contamination of mycoplasma using MycoAlert PLUS Mycoplasma Detection Kit (Lonza).

Plasmid DNA: shRNAs were cloned into lentiviral pHIV7-GFP vector. The sequences for shRNAs include control shRNA (5′-ACT CAA AAG GAA GTG ACA AGA-3′) (SEQ ID NO: 1), METTL3 shRNA-1 (5′-GCT GCA CTT CAG ACG AAT T-3′) (SEQ ID NO: 2) (Zhao et al., 2014), METTL3 shRNA-2 (5′-CCA CCT CAG TGG ATC TGT T-3′) (SEQ ID NO: 3) (Dominissini et al., 2012), METTL14 shRNA-1 (5′-GCT AAA GGA TGA GTT AAT-3′) (SEQ ID NO: 4), METTL14 shRNA-2 (5′-GGA CTT GGG ATG ATA TTA T-3′) (SEQ ID NO: 5) (Ping et al., 2014), ADAM19 shRNA-1 (5′-GGA AGA TTT AAA CTC CAT GAA G-3′) (SEQ ID NO: 6), ADAM19 shRNA-2 (5′-CAA AGT GTT CAA TGG ATG CAA C-3′) (SEQ ID NO: 7). The METTL3-expressing lentiviral vector was prepared by subcloning the human METTL3 coding sequences from pcDNA3/Flag-METTL3 (Liu et al., 2014) (Addgene plasmid #53739) into the CSC lentiviral vector (Shi et al., 2004). The METTL3 catalytic mutant (aa395-398, DPPW/APPA)-expressing lentiviral vector was prepared by subcloning the mutant human METTL3 sequences from pFLAG-CMV2-METTL3 (mutant) vector (Lin et al., 2016) into the CSC lentiviral vector.

Viral preparation and transduction: Lentiviruses were prepared using 293T cells as described (Shi et al., 2004). To transduce GSCs, cells were dissociated for overnight culture and then incubated with lentivirus and 4 μg ml⁻¹ polybrene (AmericanBio) for 24 h.

Immunostaining: GSC immunostaining was performed as previous described (Cui et al., 2016) using antibodies including mouse anti-GFAP (1:1,000; Sigma; Catalogue #G3893) and rabbit anti-Tuj1 (1:6,000; Covance; Catalogue #PRB-435P).

m⁶A dot blot assay: GSCs were either maintained in sphere cultures or induced into differentiation for 1 week using 0.5% fetal bovine serum (Sigma) together with 1 μM all-trans retinoic acid (Sigma). Total RNAs were isolated from these GSCs using Trizol reagent (Ambion). GSCs were transduced with lentivirus expressing METTL3 or METTL14 shRNA or METTL3 cDNA. Seven days after transduction, total RNAs were extracted. mRNAs were prepared from total RNAs using Dynabeads® mRNA purification kit (Ambion, Catalog #61006). Indicated amount of mRNAs was used for dot blot analysis using an antibody specific for m⁶A (1:1,000; Synaptic Systems; Catalog #202003). The intensity of dot blot signal was quantified by Image J.

RT-PCR: Total RNAs isolated using Trizol reagent (Ambion) were subjected to reverse transcription (RT) performed using the Tetro cDNA synthesis Kit (BioLINE). RT-PCR reactions were performed using SYBR Green Master Mix (Thermo Scientific) on the Step One Plus Real-Time PCR instrument (Applied Biosystems). The primers for RT-PCR include METLL3 (F 5′-TCA GCA TCG GAA CCA GCA AAG-3′ (SEQ ID NO: 8); R 5′-TCC TGA CTG ACC TTC TTG CTC-3′ (SEQ ID NO: 9)), METLL14 (F 5′-GTT GGA ACA TGG ATA GCC GC-3′ (SEQ ID NO: 10); R 5′-CAA TGC TGT CGG CAC TTT CA-3′ (SEQ ID NO: 11)), CD44 (F 5′-TGA GCA TCG GAT TTG AGA CC-3′ (SEQ ID NO: 12); R 5′-TGT CAT ACT GGG AGG TGT TGG-3′ (SEQ ID NO: 13)), ADAM19 (F 5′-CCT GGA TGG ACA AGA GGA AG-3′ (SEQ ID NO: 14); R 5′-CTC AGC TTT GAG TGG ATG CT-3′ (SEQ ID NO: 15)), EPAH3 (F 5′-GGG CTG GAT CTC TTA TCC ATC-3′ (SEQ ID NO: 16); R 5′-GGA CCC AGT TTG TTC TCA GC-3′ (SEQ ID NO: 17)), KLF4 (F 5′-AAG AGT TCC CAT CTC AAG GC-3′ (SEQ ID NO: 18); R 5′-CCG TGT GTT TAC GGT AGT GC-3′ (SEQ ID NO: 19)), and ACTIN (F 5′-CCG CAA AGA CCT GTA CGC CAA C-3′ (SEQ ID NO: 20); R 5′-CCA GGG CAG TGA TCT CCT TCT G-3′ (SEQ ID NO: 21)). ACTIN was included as the reference gene for normalization. The ΔΔCt method was used for quantification analysis.

Cell growth assay: GSCs were transduced with lentivirus expressing METTL3 or METTL14 shRNA or METTL3 cDNA. Three days later, the transduced cells were seeded at 5×10⁴ cells per well in 24-well plates and cultured for 7 days. Cell number was counted using a hemocytometer.

Sphere formation assay: The sphere formation assay was performed as previously described (Cui et al., 2016). Briefly, three days after viral transduction, the transduced cells were seeded at 1 cell per well in 96-well plates (for those associated with the limiting dilution assay) or 100 cells per well in 48-well plates and cultured for 2 weeks. The sphere number was counted under microscope. The sphere formation rate was defined as the percentage of sphere-forming cells out of the number of starting cells.

Limiting dilution assay: GSCs were transduced with relevant shRNA or METTL3-expressing lentivirus. The transduced GSCs were seeded at 1, 5, 10, 20, 50 and 100 cells per well into 96-well plates. The number of neurospheres in each well was counted two weeks after seeding cells. Extreme limiting dilution analysis was performed using software available at http://bioinf.wehi.edu.au/software/elda.

Treatment of GSCs with the FTO inhibitor MA2: GSCs were seeded at 5×10⁴ cells per well in 48-well plates and cultured overnight. These cells were treated with MA2, a chemical inhibitor of FTO (Huang et al., 2015) at 20, 40, 60, 80 μM or vehicle control and cultured for 48 h. Cell number was counted using a hemocytometer. To determine the effect of MA2 on the level of m⁶A RNA modification, GSCs were treated with 50 μM MA2 for 48 h. mRNAs were prepared and subjected to m⁶A dot blot assay. To test the effect of MA2 in limiting dilution assay, GSCs were treated at 40 μM or vehicle control for two weeks.

Animals: All animal-related work was performed under the IACUC protocol 05050 approved by the City of Hope Institutional Animal Care and Use Committee. The 6-8-week-old male and female NSG mice (from the Jackson Laboratory) were used at age- and gender-matched manner. The sample size was determined based on using t-test for two-group independent samples to reach power of 0.8 and the significance level of 0.05. p<0.05 was considered statistically significant.

Viral transduction followed by transplantation: PBT707, PBT003 or PBT726 cells expressing luciferase gene were transduced with lentivirus expressing control shRNA, METTL3 or METTL14 shRNA. One week after virus transduction, cells were transplanted into the frontal lobes of brains of NSG mice by stereotaxic intracranial injection. Briefly, 2×10⁵ dissociated cells in 2 μl PBS were injected into the following site (AP+0.6 mm, ML+1.6 mm and DV−2.6 mm) with a rate of 1 μl min⁻¹. Tumor growth was monitored by bioluminescence xenogen imaging every other week for six to ten weeks. The bioluminescence intensity was quantified. When monitoring tumor growth, investigators were blind to the group allocation during the bioluminescence xenogen imaging and aware of group allocation when assessing the outcome. The survival of mice after cell transplantation was recorded and analyzed.

Introcranial delivery of the FTO inhibitor MA2: PBT003 cells (2×10⁵) transduced with luciferase expressing lentivirus were intracranially transplanted into the frontal lobe of NSG mice at the same coordinates as described above. One week after transplantation, tumors were detected by bioluminescence imaging and mice were treated with MA2 (5 μl of 600 μM MA2 in 1% DMSO in PBS per mouse) or vehicle control by intratumoral injection once a week for four weeks. Tumor growth was monitored by bioluminescence imaging every week for six weeks. The bioluminescence intensity was quantified.

m⁶A-seq and data analysis: PBT003 cells were transduced with lentivirus expressing control shRNA or relevant shRNA. Seven days after transduction, total RNAs were extracted using Trizol reagent (Ambion). mRNA was further purified using Dynabeads® mRNA purification kit (Ambion, Catalog #61006). Fragmented RNA was subjected to m⁶A-immunoprecipitation (m⁶A IP) using anti-m⁶A rabbit polyclonal antibody (Synaptic Systems; Catalog #202003) followed by RNA-sequencing.

Purified mRNA samples from PBT003 cells were used for m⁶A-seq. RNA fragmentation was performed by sonication at 10 ng μl⁻¹ in 100 μl RNase-free water using Bioruptor Pico (Diagenode) with 30 s on/30 s off cycle for 30 cycles. m⁶A-immunoprecipitation (m⁶A IP) and library preparation were performed according to a published protocol (Dominissini et al., 2013). In detail, 2.5 μg affinity purified anti-m⁶A rabbit polyclonal antibody (Synaptic Systems; Catalog #202003) and 20 μl Protein A beads (ThermoFisher; Catalog #10002D) were used for each affinity pull-down. m⁶A antibody-bound RNAs were eluted with 100 μl elution buffer and recovered by RNA Clean and Concentrator-5 (Zymo), and subjected to RNA library preparation with TruSeq Stranded mRNA Library Prep Kit. Sequencing was carried out on Illumina HiSeq 4000 according to the manufacturer's instructions.

m⁶A seq samples (inputs and m⁶A-IPs) were sequenced by Illumina HiSeq 4000 with single end 50-bp read length. The adapters were trimmed by using the FASTX-Toolkit (Pearson et al., 1997). Deep sequencing data were mapped to Human genome version hg38 using Tophat version 2.0 (Trapnell et al., 2009) without any gaps and allowed for at most two mismatches. Input samples were analyzed by Cufflink (v2.2.1) (Trapnell et al., 2010) to generate RPKM (reads per kilobase, per million reads). For input and m⁶A-IP samples, the longest isoform was used if the gene had multiple isoforms. Aligned reads were extended to 150 bp (average fragments size) and converted from genome-based coordinates to isoform-based coordinates, in order to eliminate the interference from introns in peak calling. The peak calling method was modified from published work (Dominissini et al., 2012). To call m⁶A peaks, the longest isoform of each gene was scanned using a 100 bp sliding window with 10 bp step. To reduce bias from potential inaccurate gene structure annotation and the arbitrary usage of the longest isoform, windows with read counts less than 1/20 of the top window in both m⁶A-IP and input sample were excluded. For each gene, the read counts in each window were normalized by the median count of all windows of that gene. A Fisher exact test was used to identify the differential windows between m⁶A-IP and input samples. The window was called as positive if the FDR<0.01 and log2 (Enrichment Score)≥1. Overlapping positive windows were merged. The following four numbers were calculated to obtain the enrichment score of each peak (or window): reads count of the m⁶A-IP samples in the current peak/window (a), median read counts of the m⁶A-IP sample in all 100 bp windows on the current mRNA (b), reads count of the input sample in the current peak/window (c), and median read counts of the input sample in all 100 bp windows on the current mRNA (d). The enrichment score of each window was calculated as (a×d)/(b×c). The enrichment ratio was calculated as the ratio of enrichment score in two samples. For motif analysis, HOMER (Heinz et al., 2010) was used to search motifs in each set of m⁶A peaks. The longest isoform of all genes was used as background. For m⁶A peak distribution analysis, the length of the 5′ UTR, CDS, and 3′ UTR of each gene was normalized into 50 bins, and the normalized peak density in each bin was calculated as the percentage of gene that has m⁶A peak in that bin. The gene ontology analysis was performed using the DAVID database with biological process classified under default settings.

The accession number for the RNA-seq data presented in this paper is: GSE94808.

Western blot analysis: Twenty μg proteins from the whole cell lysates were used for Western blot analysis. Rabbit anti-METTL3 antibody (1:1000; Proteintech; Catalog #15073-1-AP), rabbit anti-METTL14 antibody (1:500; Sigma; Catalog #HPA038002), and rabbit anti-GAPDH antibody (1:1000; Santa Cruz Biotechnology; Catalog #sc-25778) were used.

Statistics: Statistical significance was analyzed using the unpaired one-tailed Student's t-test. Values were presented as *p<0.05, **p<0.01, ***p<0.001. Error bars are s.d. of the mean if not stated otherwise. Log-rank test was used for animal survival analysis.

### Example 1: m⁶A Level in GSCs Is Elevated Upon Induced Differentiation

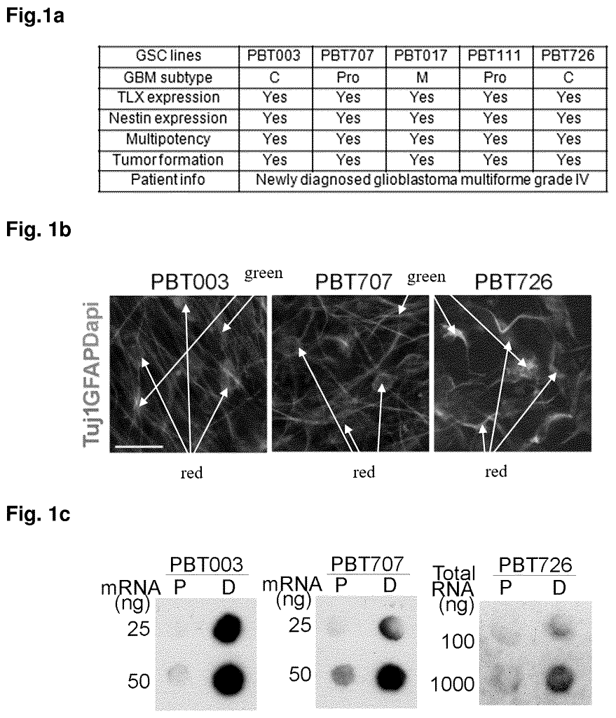

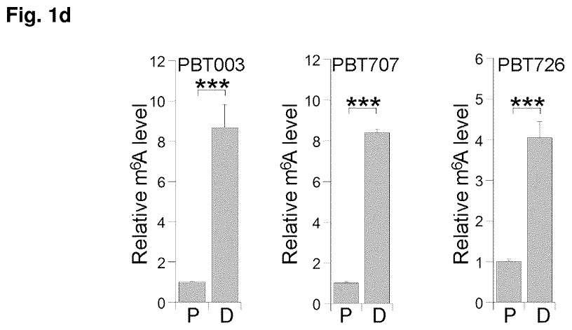

Primary GSCs were isolated from tumor tissues of newly diagnosed WHO grade IV glioblastoma patients and cultured as 3D tumorspheres in a culture condition optimized for GSC enrichment (Brown et al., 2009). Five GSC lines that represent different glioblastoma subtypes were included in this study. Among these GSC lines, PBT003 and PBT726 are classical (C), PBT707 and PBT111 are proneural (P), and PBT017 is mesenchymal (M) (Cui et al., 2016). These GSCs expressed neural stem cell markers, exhibited multipotency, having the ability to give rise to both neurons and astrocytes. Moreover, they could form brain tumors with typical glioblastoma features in transplanted mouse brains as we described previously (Cui et al., 2016). A summary of these cell lines is included in the table in FIG. 1a. To determine the relationship between cellular differentiation of GSCs and m⁶A modification, three lines of GSCs, PBT003, PBT707 and PBT726, were induced into differentiation using fetal bovine serum (FBS) together with retinoic acid as previously described (Lang et al., 2012). The differentiation of GSCs into neurons and astrocytes was confirmed by immunostaining using antibodies specific for the neuronal marker βIII tubulin (Tuj1), and the astrocyte marker GFAP (FIG. 1b). The level of m⁶A in differentiated (D) cells was measured by m⁶A mRNA dot blot, and compared to that in proliferating (P) GSCs. Dramatically elevated m⁶A level was detected in GSCs that were induced into differentiation, compared to GSCs that were proliferating (FIGS. 1c, 1d). These results indicate that m⁶A levels are dynamically regulated when GSCs are induced into differentiation.

### Example 2: Knockdown of METTL3 or METTL14 Enhances GSC Growth and Self-Renewal

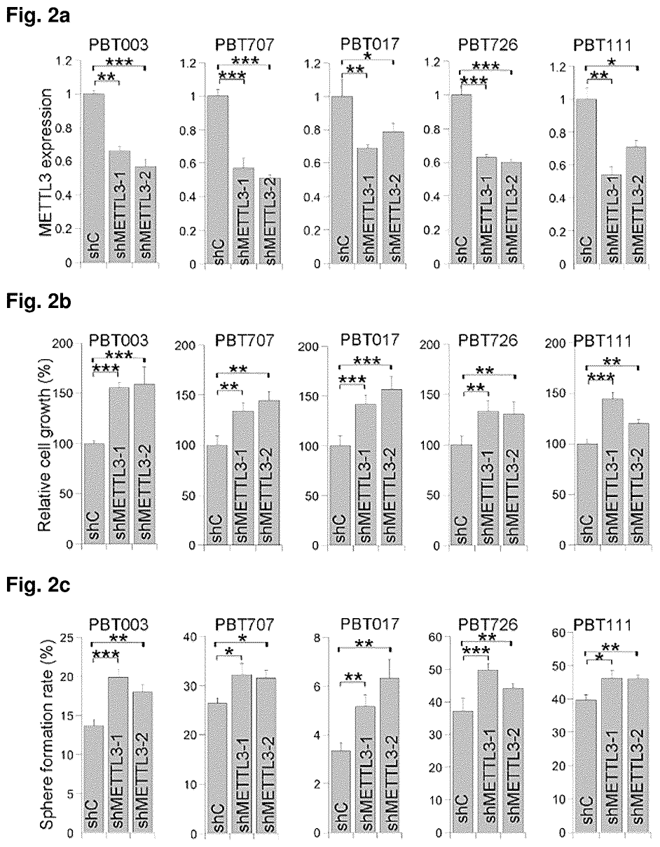

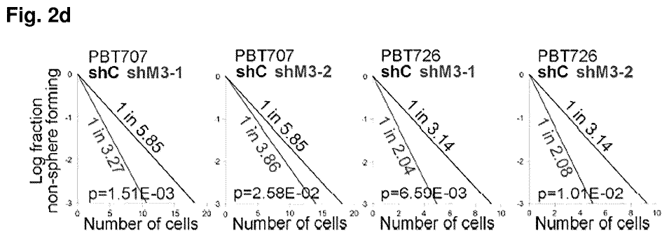

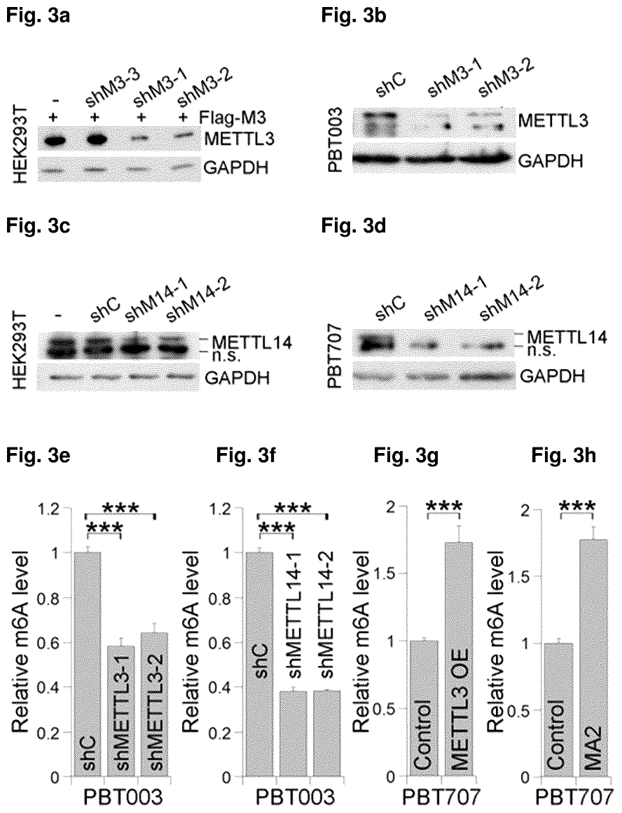

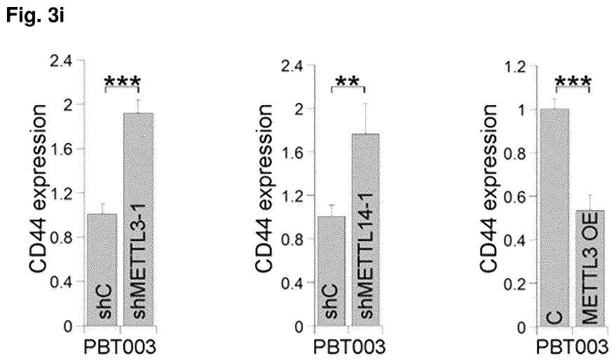

To determine if m⁶A modification plays a role in GSC self-renewal and tumorigenesis, METTL3, the catalytic subunit of m⁶A methyltransferase complex (Bokar et al., 1997; Batista et al., 2014; Liu et al., 2014; Wang et al., 2014; Geula et al., 2015), was knocked down using two distinct shRNAs in the five lines of GSCs, PBT003, PBT707, PBT017, PBT726 and PBT111. Knockdown of METTL3 expression by both shRNAs was confirmed by RT-PCR (FIG. 2a) and Western blot (FIG. 3). Reduced mRNA m⁶A level in METTL3 knockdown cells was confirmed by mRNA dot blot (FIG. 3). Knockdown of METTL3 increased cell growth substantially in all GSC lines tested (FIG. 2b). Moreover, Knockdown of METTL3 enhanced the self-renewal of these GSC lines considerably, as revealed by the significantly increased sphere formation rate and stem cell frequency in METTL3 knockdown GSCs (FIGS. 2c, 2d). Accordingly, the expression of CD44, a GSC marker (Anido et al., 2010; Pietras et al., 2014), was up-regulated in METTL3 knockdown GSCs (FIG. 2).

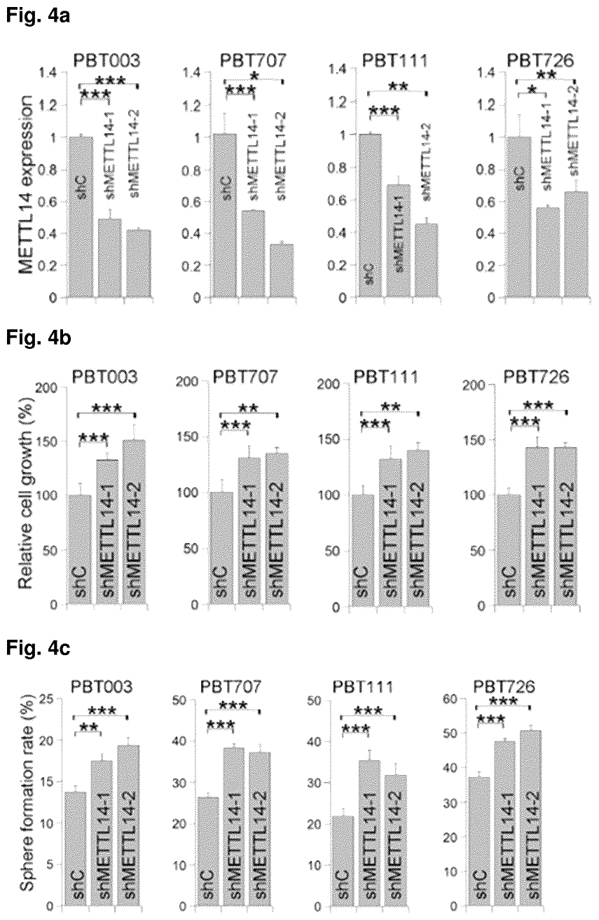

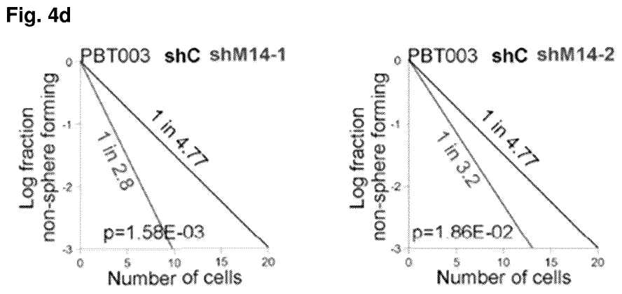

METTL14 is another component of the methyltransferase complex that is critical for m⁶A methylation (Liu et al., 2014; Ping et al., 2014; Schwartz et al., 2014; Wang et al., 2014). Like METTL3 knockdown, METTL14 knockdown has also been shown to reduce mRNA m⁶A level (Liu et al., 2014; Schwartz et al., 2014; Wang et al., 2014). To further determine the role of mRNA m⁶A methylation in GSCs, mRNA m⁶A level was modified by knocking down METTL14 using two distinct METTL14 shRNAs in GSCs, including PBT003, PBT707, PBT111 and PBT726. knockdown of METTL14 was confirmed by RT-PCR (FIG. 4a) and Western blot (FIG. 3), and reduced mRNA m⁶A level in METTL14 knockdown cells was revealed by mRNA dot blot (FIG. 3). Similar to knockdown of METTL3, knockdown of METTL14 elevated CD44 expression (FIG. 3) and enhanced the growth and self-renewal of GSCs substantially (FIGS. 4b-d). These results together indicate that reduced level of mRNA m⁶A modification promotes GSC growth and self-renewal.

### Example 3: Overexpressing METTL3 Inhibits GSC Growth and Self-Renewal

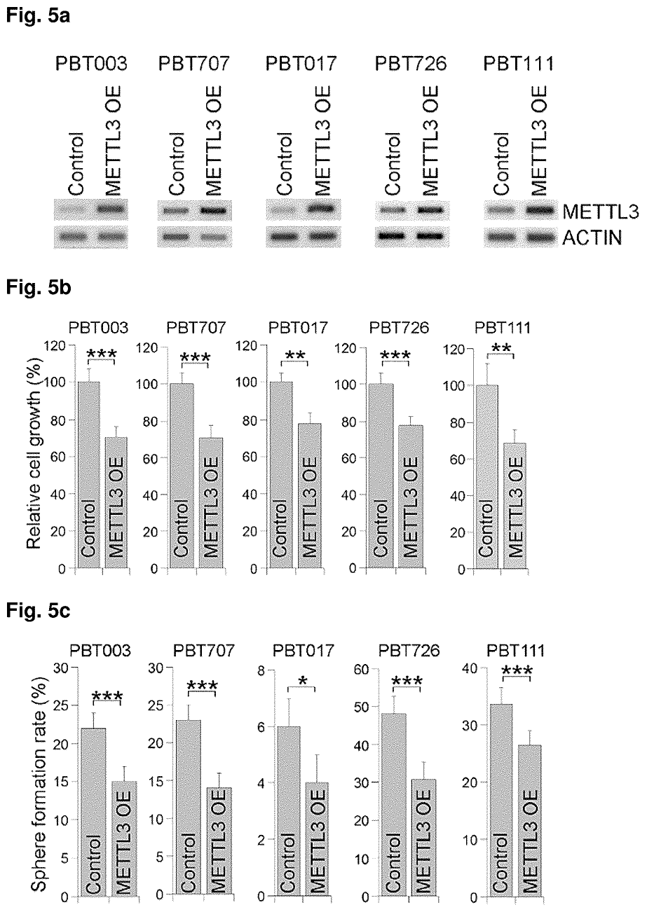

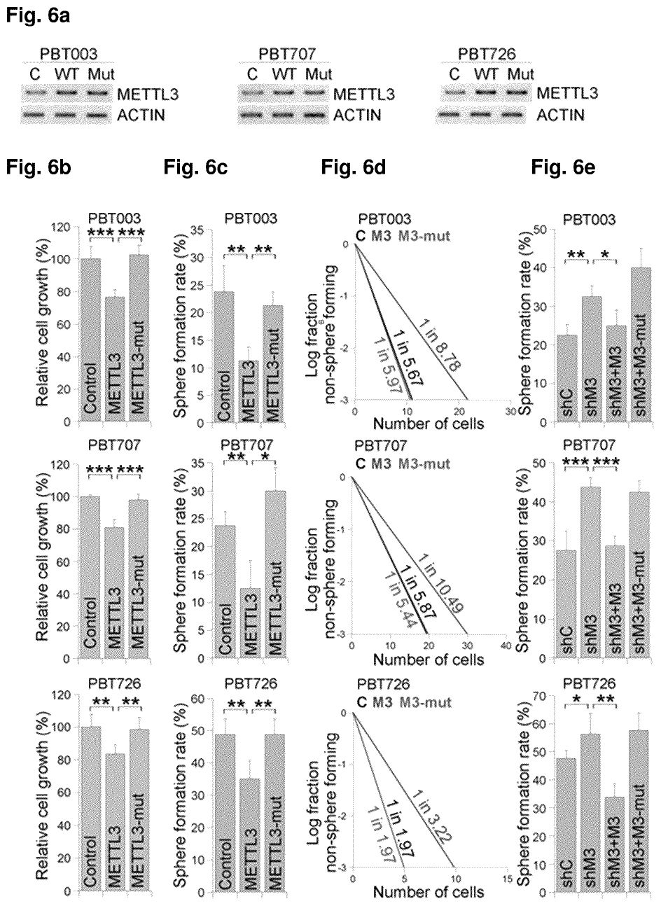

In addition to decreasing m⁶A level by knockdown of METTL3 or METTL14, how increased m⁶A level affects the growth and self-renewal of GSCs was investigated by overexpressing METTL3. Multiple GSC lines, including PBT003, PBT707, PBT017, PBT726 and PBT111, were transduced with lentivirus expressing control vector or METTL3. mRNAs were isolated from control or METTL3-overexpressing cells, and RT-PCR analysis confirmed the overexpression of METTL3 in GSCs transduced with METTL3-expressing lentivirus (FIG. 5a). Elevated m⁶A level was detected in METTL3 overexpressing GSCs as expected (FIG. 3). Overexpression of METTL3 reduced the growth and self-renewal in all GSC lines tested (FIGS. 5b, 5c). Reduced expression of CD44 was also observed in METTL3-overexpressing GSCs (FIG. 3). In contrast, overexpression of a catalytically inactive mutant of METTL3 (Lin et al., 2016) had minimal effect on GSC growth and self-renewal (FIGS. 6a-6d). Moreover, expression of the catalytically inactive METTL3 failed to reverse the elevated sphere formation phenotype induced by METTL3 knockdown, whereas expression of the wild type METTL3 was able to rescue the phenotype (FIG. 6e). These results together indicate that METTL3 regulates GSC growth and self-renewal through its methyltransferase catalytic activity.

### Example 4: Knockdown of METTL3 or METTL 14 Promotes Tumor Progression

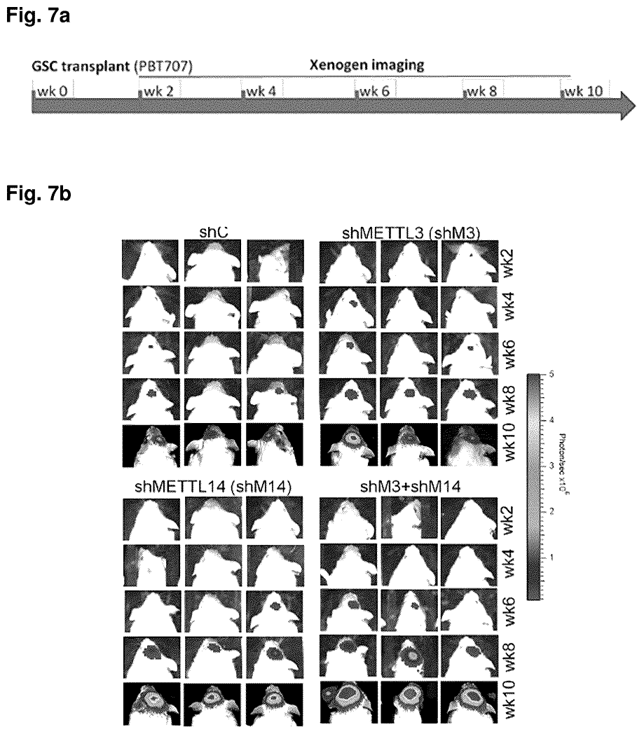

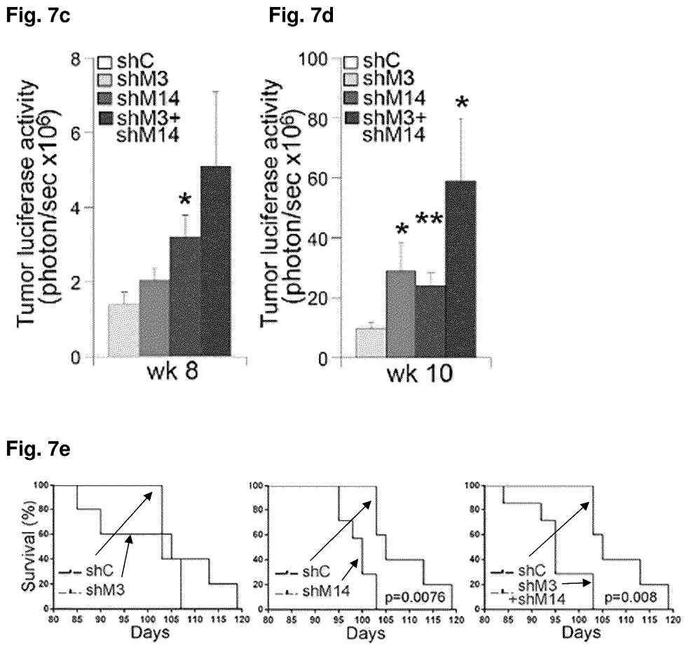

The dramatic effect of METTL3 or METTL14 KD on GSC growth and self-renewal in vitro led to the test whether knockdown of METTL3 or METTL14 affects the ability of GSCs to form tumors in vivo. The luciferase-expressing PBT707 cells were transduced with lentivirus expressing a control shRNA, a METTL3 shRNA, or a METTL14 shRNA. The transduced cells were orthotopically transplanted into the frontal lobe of NSG mouse brains (FIG. 7a). Tumor formation was monitored by bioluminescence xenogen imaging (FIG. 7b). Compared to mice receiving control shRNA-transduced GSCs (control GSCs), mice grafted with METTL3 knockdown GSCs exhibited much bigger tumors, as revealed by a substantial increase of tumor bioluminescence intensity (FIGS. 7b-7d). Likewise, mice grafted with METTL14 knockdown GSCs also exhibited dramatically bigger tumors than that in mice grafted with control GSCs (FIGS. 7b-d). In addition to single knockdown of METTL3 or METTL14, PBT707 cells were transduced with a mixture of lentivirus containing both METTL3 shRNA and METTL14 shRNA at a dose, when combined, similar to that in the single knockdown experiments. Mice grafted with the GSCs with knockdown of both METTL3 and METTL14 resulted in an even more dramatic increase in tumor progression than that in mice grafted with GSCs having METTL3 or METTL14 KD alone (FIGS. 7b-7d).

Consistent with the aggressive tumor progression, mice grafted with PBT707cells with knockdown of METTL3 or METTL14 alone, or knockdown of both METTL3 and METTL14 had considerably worse survival outcome compared to mice grafted with control GSCs (FIG. 7e). These results together indicate that inhibition of m⁶A RNA methylation by knocking down METTL3 and/or METTL14 promotes tumor progression and shortens the lifespan of GSC-grafted animals.

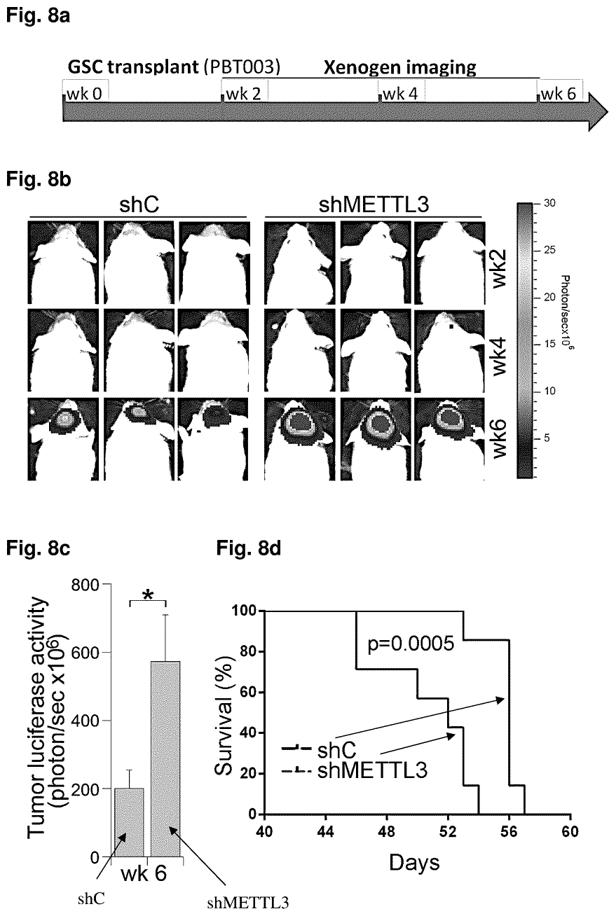

Similar to what was observed for the PBT707 cell line, when the luciferase-expressing PBT003 cells were transduced with METTL3 shRNA then transplanted into the brains of NSG mice (FIG. 8a), a dramatic increase in tumor progression as revealed by elevated tumor luciferase activity was detected, compared to that in mice transplanted with PBT003 cells transduced with a control shRNA (FIGS. 8b, 8c). Moreover, mice transplanted with METTL3 knockdown PBT003 cells exhibited overall shorter lifespan than mice transplanted with control PBT003 cells (FIG. 8d).

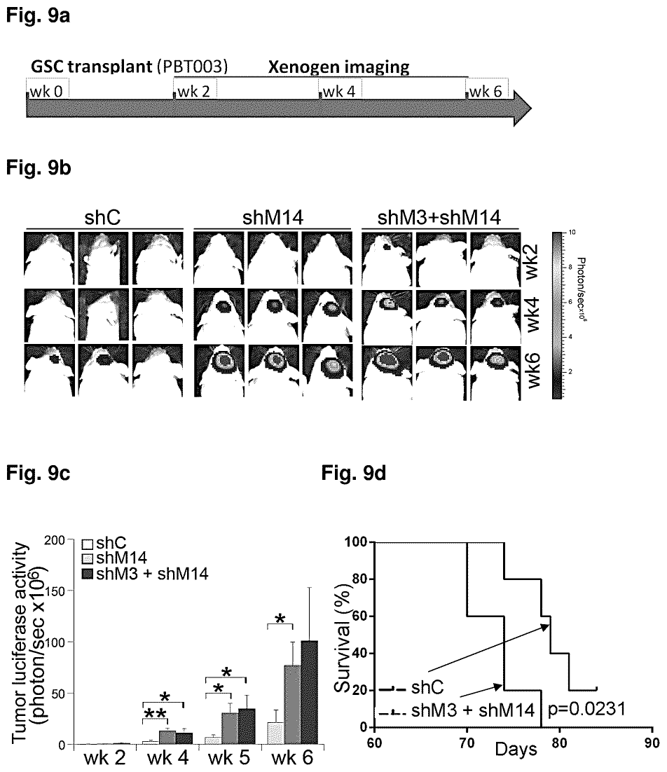

In addition to knockdown of METTL3, PBT003 cells were also transduced with lentivirus expressing a METTL14 shRNA or the combination of METTL3 and METTL14 shRNAs. The transduced cells were transplanted into the brains of NSG mice and tumor progression was monitored by xenogen imaging (FIG. 9a). Compared to mice grafted with control cells, mice transplanted with PBT003 cells having METTL14 KD or METTL3 and METTL14 double knockdown developed much bigger tumors, as revealed by a dramatic increase in tumor luciferase activity at week 4, 5 or 6 after GSC transplantation (FIGS. 9b, 9c). Moreover, mice transplanted with PBT003 cells with both METTL3 and METTL14 knockdown exhibited significantly worse survival outcome with much shorter overall lifespan, compared to mice transplanted with control cells (FIG. 9d).

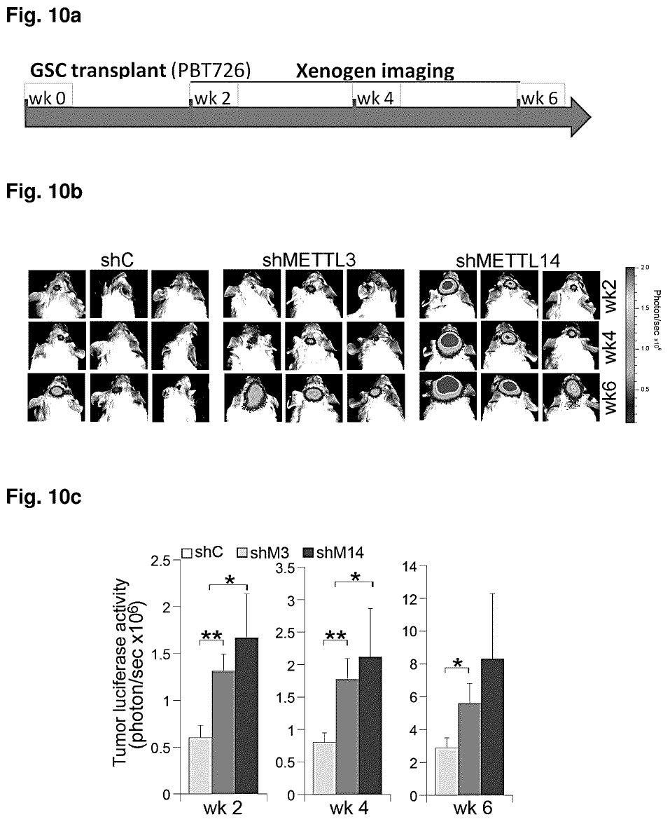

Likewise, when PBT726 cells with knockdown of METTL3 or METTL14 were transplanted into the brains of NSG mice, a substantial increase in tumor growth was observed, compared to that in mice transplanted with control cells (FIG. 10). These results further support the idea that inhibition of m⁶A methylation in GSCs by knocking down METTL3 or METTL14 promotes tumor progression.

### Example 5: The FTO Inhibitor MA2 Inhibits Tumor Progression

To evaluate the efficacy of modifying m⁶A level on GSC tumorigenesis in a more clinically relevant system, luciferase reporter-bearing PBT003 cells were transplanted into brains of NSG mice to establish tumors and the mice were treated with an FTO inhibitor that has been shown to modulate mRNA m⁶A level (Huang et al., 2015). FTO was identified as the first RNA demethylase that oxidatively demethylates m⁶A in mRNAs (Jia et al., 2011). MA2, the ethyl ester form of meclofenamic acid (MA), an FDA approved non-steroidal anti-inflammatory drug, was recently identified as a selective inhibitor of FTO that increases m⁶A level in mRNA of human cells (Huang et al., 2015). Indeed, a substantial increase in mRNA m⁶A level was detected in GSCs treated with 50 μM MA2, as shown by m⁶A mRNA dot blot analysis (FIG. 3).

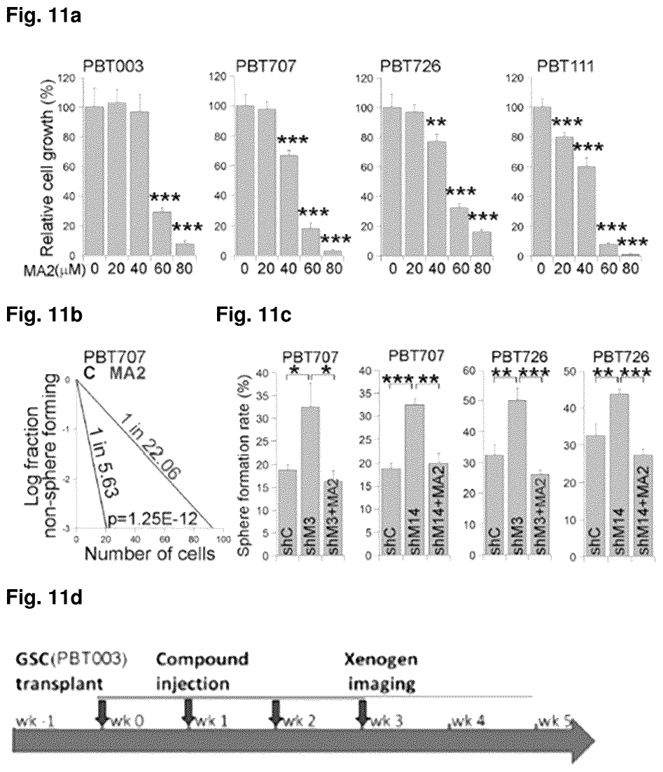

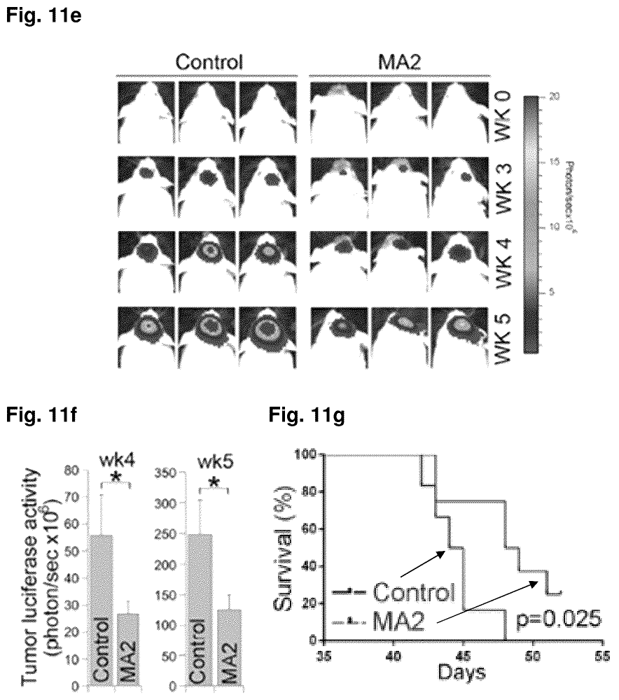

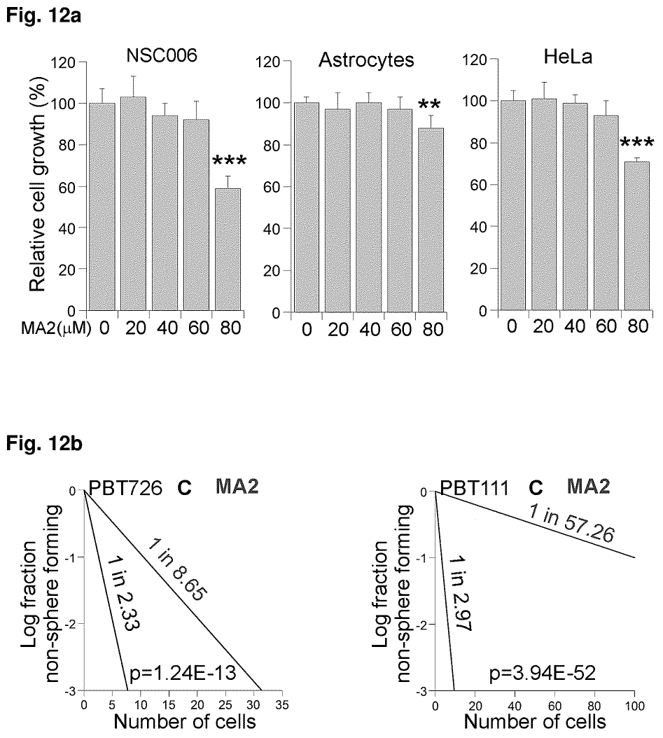

Next, the effect of MA2 treatment on GSC growth and self-renewal was tested in vitro. A dramatic growth inhibitory effect of MA2 was detected in PBT003 cells at 60 μM and 80 μM doses (FIG. 11a). MA2 also exerted substantial inhibitory effect on the growth of other GSC lines, including PBT707, PBT726 and PBT111, at the same doses (FIG. 11a). In addition, MA2 suppressed the growth of PBT707 and PBT726 at a lower dose of 40 μM and inhibited the growth of PBT111 at lower doses of 20 μM and 40 μM (FIG. 11a). In contrast, no substantial effect was detected on the growth of the normal neural stem cells (NSC) line NSC006, brain astrocytes, or HeLa cells, by MA2 at a dosage up to 60 μM (FIG. 12a). Mild effect was observed in NSCs, astrocytes or HeLa cells treated with 80 μM MA2 (FIG. 12a). In addition to suppression of GSC growth, MA2 treatment dramatically inhibited the self-renewal of GSCs as revealed by reduced stem cell frequency in MA2 treated GSCs, compared to that in control cells (FIG. 11b and FIG. 12b). Moreover, MA2 treatment reversed the effect of elevated sphere formation rate induced by METTL3 or METTL14 KD in GSCs (FIG. 11c). The results of MA2 treatment corroborated our observation in GSCs with overexpression of METTL3, further strengthening the hypothesis that increase of m⁶A level inhibits GSC growth and self-renewal.

To test the effect of the FTO inhibitor on GSC-initiated tumorigenesis, PBT003-grafted mice were treated with the selective FTO inhibitor MA2 intratumorally once a week for four weeks (FIG. 11d). Tumor formation was monitored by bioluminescence xenogen imaging. Compared to mice receiving vehicle control, mice treated with MA2 had much smaller tumors (FIG. 11e). Bioluminescence measurement showed a significant decrease of tumor luciferase activity in mice treated with MA2 at 4 or 5 weeks after compound treatment (FIG. 11f). Consistent with reduced tumor growth, mice treated with MA2 had substantially prolonged survival compared to mice treated with vehicle control (FIG. 11g). This result indicates that small molecule compounds that increase m⁶A RNA methylation have therapeutic potential to inhibit GSC tumorigenesis.

### Example 6: Knockdown of METTL3 or METTL 14 Alters Gene Expression in GSCs

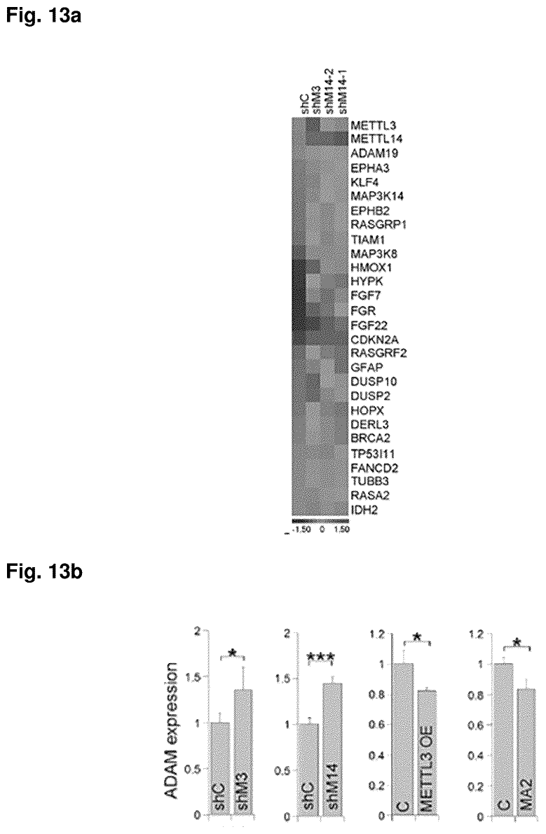

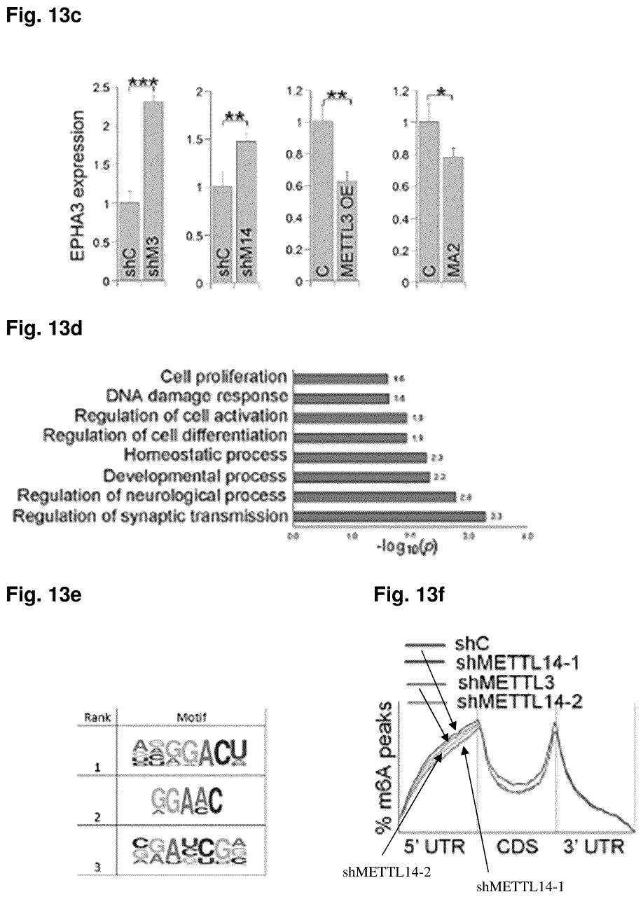

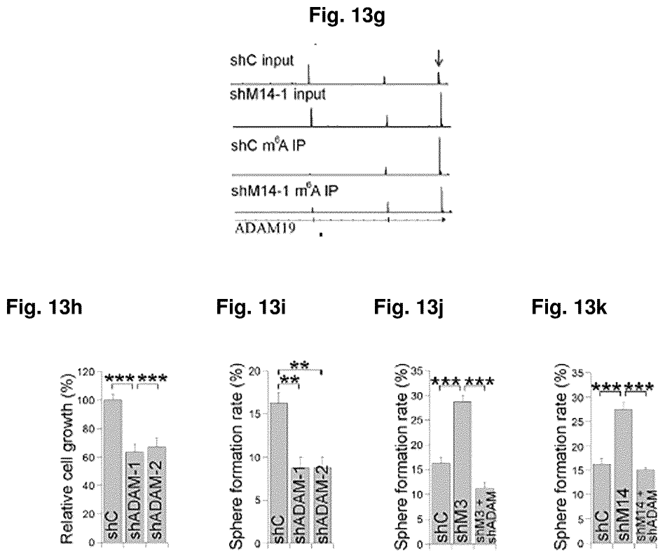

To investigate the mechanism underlying how m⁶A modification regulates GSC tumorigenesis, RNA-seq was performed to detect gene expression changes in PBT003 cells with knockdown of METTL3 or METTL14. The expression of more than 2,600 transcripts was changed in PBT003 cells with METTL3 or METTL14 knockdown compared to control cells. Among the genes with altered expression, a number of oncogenes, such as ADAM19, EPHA3, and KLF4, were up-regulated and a list of tumor suppressors, such as CDKN2A, BRCA2, and TP53111, was down-regulated in GSCs with knockdown of METTL3 or METTL14 (FIG. 13a). The expression of differentiated neural cell markers, such as the astrocyte marker GFAP and the neuronal marker TUBB3 (Tuj1), was also decreased in METTL3 or METTL14 KD GSCs (FIG. 13a). These gene expression changes were consistent with the hypothesis that knockdown of METTL3 or METTL14 promotes GSC self-renewal and tumorigenesis.

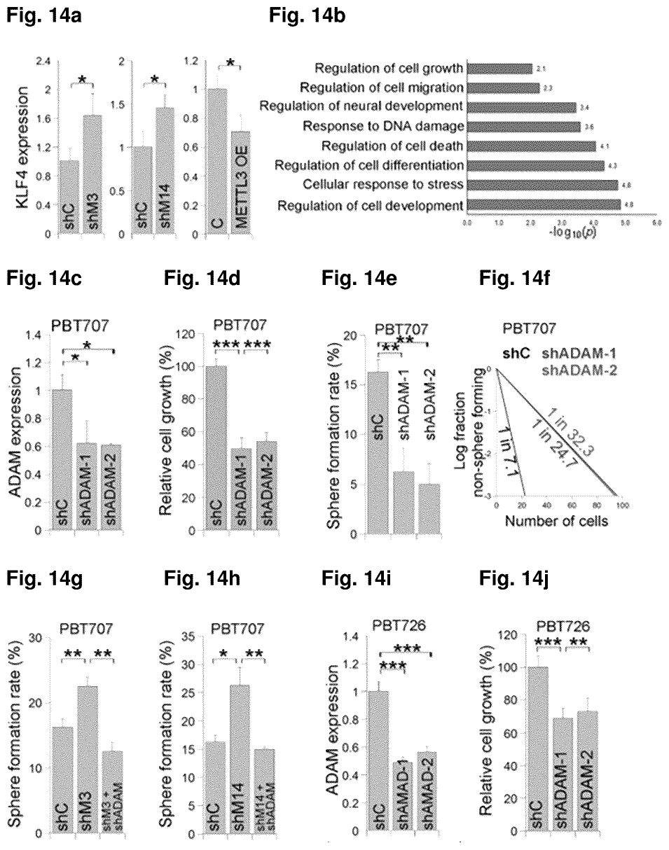

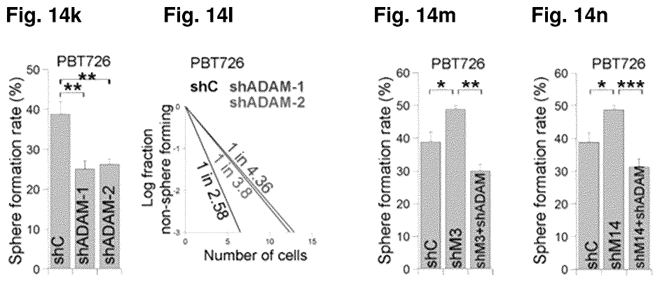

The up-regulated expression of oncogenes, such as ADAM19, EPHA3, and KLF4, in METTL3 or METTL14 knockdown GSCs was confirmed by RT-PCR. In contrast, overexpression of METTL3 or treatment with the FTO inhibitor MA2 led to decreased expression of these genes (FIGS. 13b, 13c & FIG. 14a). The altered expression of these oncogenes by perturbation of m⁶A modification suggests that the expressions of these genes are regulated by m⁶A RNA methylation. The gene ontology (GO) analysis revealed that knocking down METTL3 or METTL14 regulated the expression of genes involved in important biological processes, including cell proliferation, differentiation, and DNA damage response (FIG. 13d). Taking together, these data indicate that mRNA m⁶A modification could regulate GSC tumorigenesis through controlling the expression of cancer-associated genes and processes.

### Example 7: m⁶A-Modified mRNAs Are Involved in Critical Cellular Processes

To investigate the m⁶A modifications in GSC transcriptome, m⁶A-seq analysis was performed as described (Dominissini et al., 2013). The m⁶A consensus motif GGAC was identified in PBT003 cells (FIG. 13e). Peak distribution analysis revealed strong enrichment of m⁶A peaks near stop codon (FIG. 13f) as previously described (Dominissini et al., 2012; Meyer et al., 2012). Of interest, strong enrichment of m⁶A peaks was also detected near start codon in GSCs (FIG. 13f). The GO analysis of genes with m⁶A peaks in their mRNAs revealed that m⁶A-methylated mRNAs are involved in critical cellular processes, such as cell growth, cell differentiation, DNA damage response, and cellular stress response (FIG. 14b).

Among the genes, the mRNA of which is modified by m⁶A methylation, a list of them plays critical roles in cell growth and tumorigenesis. For example, the mRNA of ADAM19, a metalloproteinase disintegrin gene that exhibits elevated expression in glioblastoma cells and promotes glioblastoma cell growth and invasiveness (Wildeboer et al., 2006; Mochizuki and Okada, 2007), is m⁶A methylated. The mRNA expression of ADAM19 is highly elevated in METTL14 KD GSCs, as revealed by increased mRNA reads in METTL14 KD GSCs (shM14-1 input), compared to control GSCs (shC input) (FIG. 13g). In contrast, the m⁶A enrichment in the mRNA of this gene is dramatically reduced upon knockdown of METTL14, as revealed by the reduced mRNA m⁶A peak in METTL14 KD GSCs (shM14-1 m⁶A IP), compared to control GSCs (shC m⁶A IP) (FIG. 13g), correlating with up-regulated expression of this gene by knockdown of METTL14. Knockdown of ADAM19 dramatically reduced the growth and self-renewal of GSCs (FIGS. 13h, 13i & FIG. 14). Moreover, the elevated sphere formation rate induced by knockdown of METTL3 or METTL14 in GSCs could be reversed by knockdown of ADAM19 (FIGS. 13j, 13k & FIG. 14), suggesting that ADAM19 acts as a target of m⁶A RNA methylation to regulates GSC self-renewal. Taking together, these data demonstrate that mRNA m⁶A methylation is an important RNA epigenetic marker that is involved in regulating the expression of genes with important biological functions in GSCs.

### Example 8: Correlation of Elevated METTL3 and METTL 14 Levels With Survival

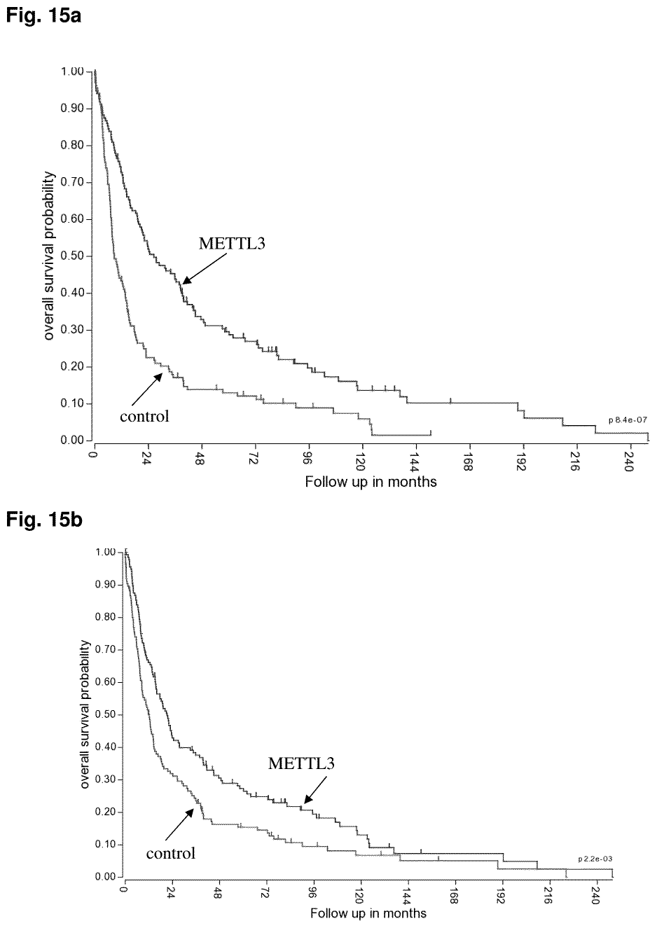

To study whether METTL3 and METTL14 are associated with the outcome of glioma patients, the expressions of METTL3 and METTL14 in the R2 French database were investigated. It was found that the upregulated expression of METTL3 and METTL14 predicts better survival in glioma patients (FIGS. 15a, 15b).

### Example 9: The Nuclear Receptor TLX Is Essential for GSC Self-Renewal and Tumorigenecity

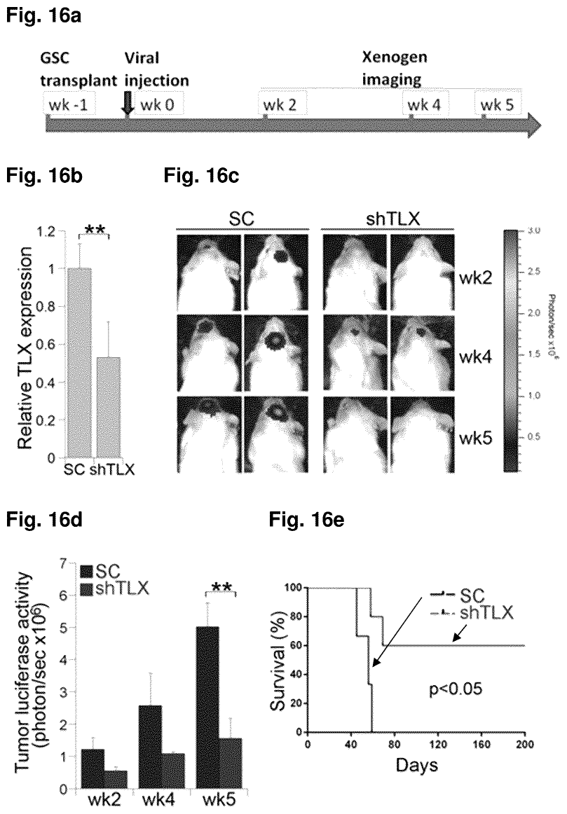

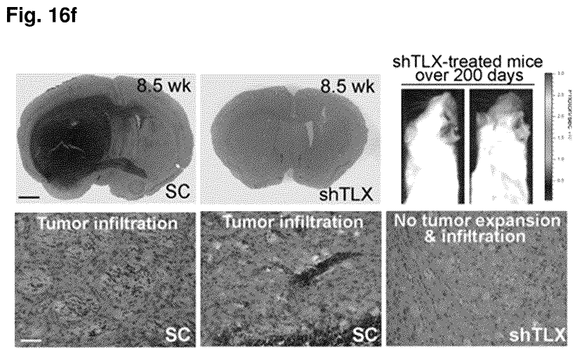

Whether knocking down TLX in vivo could suppress the progression of human GSC-initiated tumors was investigated in a xenograft model. PBT003 cells were transduced with luciferase-expressing lentivirus, which allowed monitoring tumor growth in vivo by bioluminescence imaging. The resultant PBT003 cells were orthotopically transplanted into the frontal lobe of NSG mouse brains to establish tumors. One week after, mice were treated with scrambled control RNA or TLX shRNA-expressing lentivirus by intratumoral injection (FIG. 16a). Knockdown of TLX in PBT003 cells in vivo was confirmed by RT-PCR using human TLX-specific primers (FIG. 16b). Tumor formation was monitored using bioluminescence xenogen imaging (FIG. 16c). Mice received control RNA-expressing virus developed large tumors, whereas mice treated by TLX shRNA-expressing lentivirus had much smaller tumors (FIG. 16c, 16d). Bioluminescence measurement showed a significant decrease of tumor signal in mice treated with TLX shRNA-expressing virus at 5 weeks after treatment (FIG. 16d).

Moreover, PBT003-grafted mice treated with TLX shRNA-expressing virus had much better survival outcome compared to mice treated with scrambled control RNA (FIG. 16e). All mice that received control RNA died before day 60 post-treatment and the median survival was 56 days after viral treatment, whereas 60% of mice treated with TLX shRNA survived beyond 200 days post-treatment (FIG. 16e).

When mice in control group died, brain samples were collected for histological analysis. H&E staining revealed the development of big tumor mass and aggressive tumor invasion across the hemisphere in brains of control mice, whereas in brains of TLX shRNA-treated mice collected at the same time, no or much smaller tumor was detected (FIG. 16f). The tumors developed in control mice exhibited typical infiltrative features of glioblastoma (FIG. 16f). These results indicate that TLX shRNA-expressing virus suppressed the progression of established tumors and increased the lifespan of treated animals.

### Example 10: Knockdown or Knockout of TLX Elevates m⁶A RNA Modification

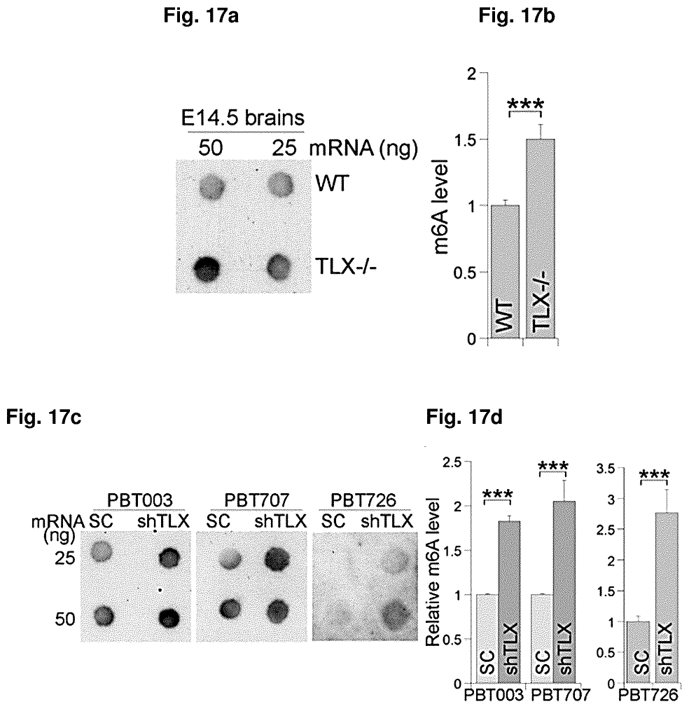

To study whether TLX regulates m⁶A RNA modification, the level of m⁶A in TLX wild type mouse brain cells was measured by m⁶A mRNA dot blot, and compared to that in TLX knockout mouse brain cells. Dramatically elevated m⁶A level was detected in cells from TLX knockout mouse brain, compared to cells from wild type mouse brain (FIGS. 17a, 17b). Consistently, the m⁶A level was elevated in GSCs with knockdown of TLX, compated to that in control GSCs (FIGS. 17c, 17d). These results indicate that knockdown or knockout of TLX elevates m⁶A RNA modification.

### Example 11: Knockdown of METTL3 Reverses TLX Knockdown-Induced Growth Inhibition in GSCs

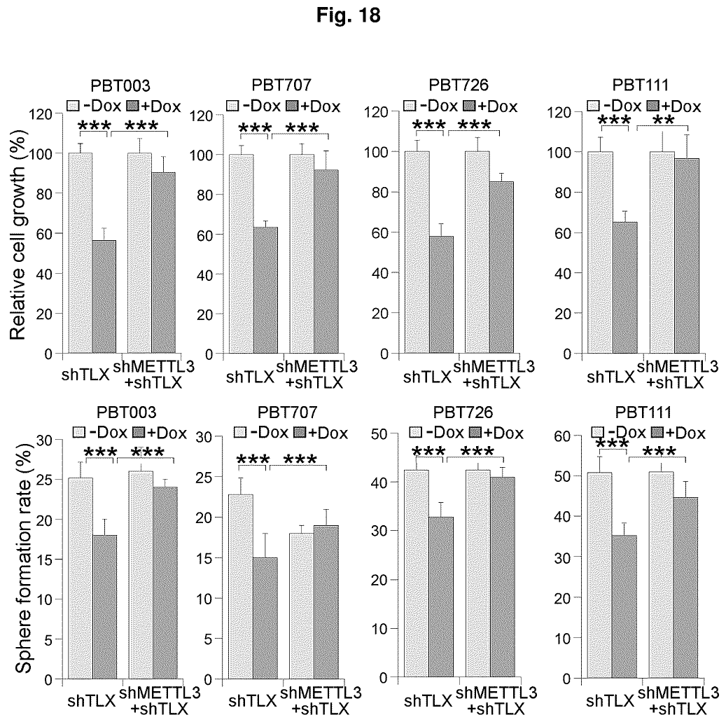

To study whether METTL3 works downstream of TLX to regulate the growth and self-renewal of GSCs, an inducible system to double knock down TLX and METTL3 was established. PBT003, PBT707, PBT726, and PBT111 cells were transduced with lentivirus that expresses doxycycline (dox)-inducible TLX shRNA alone or transduced with lentivirus that expresses dox-inducible TLX shRNA together with lentivirus that expresses dox-inducible METTL3 shRNA. Next whether inducible METTL3 knockdown could rescue the inhibitory effect of inducible TLX knockdown on GSC growth and self-renewal was tested. After dox induction, the growth of PBT003, PBT707, PBT726, and PBT111 cells expressing inducible TLX shRNA was reduced when compared to non-induced cells (FIG. 18). The decreased cell growth resulted from induced TLX knockdown was rescued substantially by induced METTL3 knockdown in PBT003, PBT707, PBT726, and PBT111 cells (FIG. 18). The reduced self-renewal of GSCs resulted from dox-induced TLX knockdown was also rescued by dox-induced METTL3 knockdown (FIG. 18). These results indicate that METTL3 is a critical downstream target of TLX in regulating GSC growth and self-renewal.

As stated above, the foregoing are merely intended to illustrate the various embodiments of the present invention. As such, the specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

Allegra, A., Alonci, A., Penna, G., Innao, V., Gerace, D., Rotondo, F., and Musolino, C. (2014). The cancer stem cell hypothesis: a guide to potential molecular targets. Cancer Invest 32, 470-495.
Anido, J., Saez-Borderias, A., Gonzalez-Junca, A., Rodon, L., Folch, G., Carmona, M.A., Prieto-Sanchez, R.M., Barba, I., Martinez-Saez, E., Prudkin, L., et al. (2010). TGF-beta Receptor Inhibitors Target the CD44 (high)/Id1 (high) Glioma-Initiating Cell Population in Human Glioblastoma. Cancer cell 18, 655-668.
Bao, S., Wu, Q., McLendon, R.E., Hao, Y., Shi, Q., Hjelmeland, A.B., Dewhirst, M.W., Bigner, D.D., and Rich, J.N. (2006). Glioma stem cells promote radioresistance by preferential activation of the DNA damage response. Nature 444, 756-760.
Batista, P.J., Molinie, B., Wang, J., Qu, K., Zhang, J., Li, L., Bouley, D.M., Lujan, E., Haddad, B., Daneshvar, K., et al. (2014). m(6)A RNA modification controls cell fate transition in mammalian embryonic stem cells. Cell stem cell 15, 707-719.
Bokar, J.A., Rath-Shambaugh, M.E., Ludwiczak, R., Narayan, P., and Rottman, F. (1994). Characterization and partial purification of mRNA N6-adenosine methyltransferase from HeLa cell nuclei. Internal mRNA methylation requires a multisubunit complex. The Journal of biological chemistry 269, 17697-17704.
Bokar, J.A., Shambaugh, M.E., Polayes, D., Matera, A.G., and Rottman, F.M. (1997). Purification and cDNA cloning of the AdoMet-binding subunit of the human mRNA (N6-adenosine)-methyltransferase. RNA (New York, NY 3, 1233-1247.
Brown, C.E., Starr, R., Martinez, C., Aguilar, B., D'Apuzzo, M., Todorov, I., Shih, C.C., Badie, B., Hudecek, M., Riddell, S.R., et al. (2009). Recognition and killing of brain tumor stem-like initiating cells by CD8+ cytolytic T cells. Cancer research 69, 8886-8893.
Chen, T., Hao, Y.J., Zhang, Y., Li, M.M., Wang, M., Han, W., Wu, Y., Lv, Y., Hao, J., Wang, L., et al. (2015). m(6)A RNA methylation is regulated by microRNAs and promotes reprogramming to pluripotency. Cell stem cell 16, 289-301.
Cui, Q., Yang, S., Ye, P., Tian, E., Sun, G., Zhou, J., Sun, G., Liu, X., Chen, C., Murai, K., et al. (2016). Downregulation of TLX induces TET3 expression and inhibits glioblastoma stem cell self-renewal and tumorigenesis. Nature communications 7, 10637.
Desrosiers, R., Friderici, K., and Rottman, F. (1974). Identification of methylated nucleosides in messenger RNA from Novikoff hepatoma cells. Proceedings of the National Academy of Sciences of the United States of America 71, 3971-3975.
Dominissini, D., Moshitch-Moshkovitz, S., Salmon-Divon, M., Amariglio, N., and Rechavi, G. (2013). Transcriptome-wide mapping of N(6)-methyladenosine by m(6)A-seq based on immunocapturing and massively parallel sequencing. Nature protocols 8, 176-189.
Dominissini, D., Moshitch-Moshkovitz, S., Schwartz, S., Salmon-Divon, M., Ungar, L., Osenberg, S., Cesarkas, K., Jacob-Hirsch, J., Amariglio, N., Kupiec, M., et al. (2012). Topology of the human and mouse m6A RNA methylomes revealed by m6A-seq. Nature 485, 201-206.
Dubin, D.T., and Taylor, R.H. (1975). The methylation state of poly A-containing messenger RNA from cultured hamster cells. Nucleic acids research 2, 1653-1668.
Fustin, J.M., Doi, M., Yamaguchi, Y., Hida, H., Nishimura, S., Yoshida, M., Isagawa, T., Morioka, M.S., Kakeya, H., Manabe, I., et al. (2013). RNA-methylation-dependent RNA processing controls the speed of the circadian clock. Cell 155, 793-806.
Geula, S., Moshitch-Moshkovitz, S., Dominissini, D., Mansour, A.A., Kol, N., Salmon-Divon, M., Hershkovitz, V., Peer, E., Mor, N., Manor, Y.S., et al. (2015). m6A mRNA methylation facilitates resolution of naive pluripotency toward differentiation. Science (New York, NY 347, 1002-1006.
Godlewski, J., Newton, H.B., Chiocca, E.A., and Lawler, S.E. (2010). MicroRNAs and glioblastoma; the stem cell connection. Cell death and differentiation 17, 221-228.
Guruswamy, S., Swamy, M.V., Choi, C.I., Steele, V.E., and Rao, C.V. (2008). S-adenosyl L-methionine inhibits azoxymethane-induced colonic aberrant crypt foci in F344 rats and suppresses human colon cancer Caco-2 cell growth in 3D culture. International journal of cancer 122, 25-30.
Haussmann, I.U., Bodi, Z., Sanchez-Moran, E., Mongan, N.P., Archer, N., Fray, R.G., and Soller, M. (2016). m6A potentiates Sxl alternative pre-mRNA splicing for robust Drosophila sex determination. Nature 540, 301-304.
Heinz, S., Benner, C., Spann, N., Bertolino, E., Lin, Y.C., Laslo, P., Cheng, J.X., Murre, C., Singh, H., and Glass, C.K. (2010). Simple combinations of lineage-determining transcription factors prime cis-regulatory elements required for macrophage and B cell identities. Molecular cell 38, 576-589.
Huang, Y., Yan, J., Li, Q., Li, J., Gong, S., Zhou, H., Gan, J., Jiang, H., Jia, G.F., Luo, C., et al. (2015). Meclofenamic acid selectively inhibits FTO demethylation of m6A over ALKBH5. Nucleic acids research 43, 373-384.
Jia, G., Fu, Y., Zhao, X., Dai, Q., Zheng, G., Yang, Y., Yi, C., Lindahl, T., Pan, T., Yang, Y.G., et al. (2011). N6-methyladenosine in nuclear RNA is a major substrate of the obesity-associated FTO. Nature chemical biology 7, 885-887.
Johnson, D.R., and O'Neill, B.P. (2012). Glioblastoma survival in the United States before and during the temozolomide era. Journal of neuro-oncology 107, 359-364.
Kaklamani, V., Yi, N., Sadim, M., Siziopikou, K., Zhang, K., Xu, Y., Tofilon, S., Agarwal, S., Pasche, B., and Mantzoros, C. (2011). The role of the fat mass and obesity associated gene (FTO) in breast cancer risk. BMC Med Genet 12, 52.
Lang, M.F., Yang, S., Zhao, C., Sun, G., Murai, K., Wu, X., Wang, J., Gao, H., Brown, C.E., Liu, X., et al. (2012). Genome-wide profiling identified a set of miRNAs that are differentially expressed in glioblastoma stem cells and normal neural stem cells. PloS one 7, e36248.
Lemkine, G.F., Raj, A., Alfama, G., Turque, N., Hassani, Z., Alegria-Prevot, O., Samarut, J., Levi, G., and Demeneix, B.A. (2005). Adult neural stem cell cycling in vivo requires thyroid hormone and its alpha receptor. Faseb J 19, 863-865.
Lence, T., Akhtar, J., Bayer, M., Schmid, K., Spindler, L., Ho, C.H., Kreim, N., Andrad-Navarro, M.A., Poeck, B., Helm, M., and Roignant, J.-Y. (2016). m⁶A medulates neuronal functions and sex determination in Drosophila. Nature Comm 000, 1-6.
Li, Z., Weng, H., Su, R., Weng, X., Zuo, Z., Li, C., Huang, H., Nachtergaele, S., Dong, L., Hu, C., et al. (2017). FTO Plays an Oncogenic Role in Acute Myeloid Leukemia as a N6-Methyladenosine RNA Demethylase. Cancer cell 31, 127-141.
Lin, S., Choe, J., Du, P., Triboulet, R., and Gregory, R.I. (2016). The m(6)A Methyltransferase METTL3 Promotes Translation in Human Cancer Cells. Molecular cell 62, 335-345.
Linnebacher, M., Wienck, A., Boeck, I., and Klar, E. (2010). Identification of an MSI-H tumor-specific cytotoxic T cell epitope generated by the (−1) frame of U79260(FTO). Journal of biomedicine & biotechnology 2010, 841451.
Liu, J., Yue, Y., Han, D., Wang, X., Fu, Y., Zhang, L., Jia, G., Yu, M., Lu, Z., Deng, X., et al. (2014). A METTL3-METTL14 complex mediates mammalian nuclear RNA N6-adenosine methylation. Nature chemical biology 10, 93-95.
Long, J., Zhang, B., Signorello, L.B., Cai, Q., Deming-Halverson, S., Shrubsole, M.J., Sanderson, M., Dennis, J., Michailidou, K., Easton, D.F., et al. (2013). Evaluating genome-wide association study-identified breast cancer risk variants in African-American women. PloS one 8, e58350.
Lu, S.C., Ramani, K., Ou, X., Lin, M., Yu, V., Ko, K., Park, R., Bottiglieri, T., Tsukamoto, H., Kanel, G., et al. (2009). S-adenosylmethionine in the chemoprevention and treatment of hepatocellular carcinoma in a rat model. Hepatology 50, 462-471.
Machiela, M.J., Lindstrom, S., Allen, N.E., Haiman, C.A., Albanes, D., Barricarte, A., Berndt, S.I., Bueno-de-Mesquita, H.B., Chanock, S., Gaziano, J.M., et al. (2012). Association of type 2 diabetes susceptibility variants with advanced prostate cancer risk in the Breast and Prostate Cancer Cohort Consortium. Am J Epidemiol 176, 1121-1129.
Machnicka, M.A., Milanowska, K., Osman Oglou, O., Purta, E., Kurkowska, M., Olchowik, A., Januszewski, W., Kalinowski, S., Dunin-Horkawicz, S., Rother, K.M., et al. (2013). MODOMICS: a database of RNA modification pathways—2013 update. Nucleic acids research 41, D262-267.
Meyer, K.D., Saletore, Y., Zumbo, P., Elemento, O., Mason, C.E., and Jaffrey, S.R. (2012). Comprehensive analysis of mRNA methylation reveals enrichment in 3′ UTRs and near stop codons. Cell 149, 1635-1646.
Mochizuki, S., and Okada, Y. (2007). ADAMs in cancer cell proliferation and progression. Cancer Sci 98, 621-628.
Pakneshan, P., Szyf, M., Farias-Eisner, R., and Rabbani, S.A. (2004). Reversal of the hypomethylation status of urokinase (uPA) promoter blocks breast cancer growth and metastasis. The Journal of biological chemistry 279, 31735-31744.
Pascale, R.M., Simile, M.M., De Miglio, M.R., and Feo, F. (2002). Chemoprevention of hepatocarcinogenesis: S-adenosyl-L-methionine. Alcohol 27, 193-198.
Pearson, W.R., Wood, T., Zhang, Z., and Miller, W. (1997). Comparison of DNA sequences with protein sequences. Genomics 46, 24-36.
Perry, R.P., Kelley, D.E., Friderici, K., and Rottman, F. (1975). The methylated constituents of L cell messenger RNA: evidence for an unusual cluster at the 5′ terminus. Cell 4, 387-394.
Pierce, B.L., Austin, M.A., and Ahsan, H. (2011). Association study of type 2 diabetes genetic susceptibility variants and risk of pancreatic cancer: an analysis of PanScan-I data. Cancer Causes Control 22, 877-883.
Pietras, A., Katz, A.M., Ekstrom, E.J., Wee, B., Halliday, J.J., Pitter, K.L., Werbeck, J.L., Amankulor, N.M., Huse, J.T., and Holland, E.C. (2014). Osteopontin-CD44 signaling in the glioma perivascular niche enhances cancer stem cell phenotypes and promotes aggressive tumor growth. Cell stem cell 14, 357-369.
Ping, X.L., Sun, B.F., Wang, L., Xiao, W., Yang, X., Wang, W.J., Adhikari, S., Shi, Y., Lv, Y., Chen, Y.S., et al. (2014). Mammalian WTAP is a regulatory subunit of the RNA N6-methyladenosine methyltransferase. Cell research 24, 177-189.
Schwartz, S., Agarwala, S.D., Mumbach, M.R., Jovanovic, M., Mertins, P., Shishkin, A., Tabach, Y., Mikkelsen, T.S., Satija, R., Ruvkun, G., et al. (2013). High-resolution mapping reveals a conserved, widespread, dynamic mRNA methylation program in yeast meiosis. Cell 155, 1409-1421.
Schwartz, S., Mumbach, M.R., Jovanovic, M., Wang, T., Maciag, K., Bushkin, G.G., Mertins, P., Ter-Ovanesyan, D., Habib, N., Cacchiarelli, D., et al. (2014). Perturbation of m6A writers reveals two distinct classes of mRNA methylation at internal and 5′ sites. Cell Rep 8, 284-296.
Shi, Y., Lie, C.D., Taupin, P., Nakashima, K., Ray, J., Yu, R.T., Gage, F.H., and Evans, R.M. (2004). Expression and function of orphan nuclear receptor TLX in adult neural stem cells. Nature 427, 78-83.
Sibbritt, T., Patel, H.R., and Preiss, T. (2013). Mapping and significance of the mRNA methylome. Wiley Interdiscip Rev RNA 4, 397-422.
Singh, S.K., Hawkins, C., Clarke, I.D., Squire, J.A., Bayani, J., Hide, T., Henkelman, R.M., Cusimano, M.D., and Dirks, P.B. (2004). Identification of human brain tumour initiating cells. Nature 432, 396-401.
Stupp, R., Hegi, M.E., Mason, W.P., van den Bent, M.J., Taphoorn, M.J., Janzer, R.C., Ludwin, S.K., Allgeier, A., Fisher, B., Belanger, K., et al. (2009). Effects of radiotherapy with concomitant and adjuvant temozolomide versus radiotherapy alone on survival in glioblastoma in a randomised phase III study: 5-year analysis of the EORTC-NCIC trial. Lancet Oncol 10, 459-466.
Sundar, S.J., Hsieh, J.K., Manjila, S., Lathia, J.D., and Sloan, A. (2014). The role of cancer stem cells in glioblastoma. Neurosurgical focus 37, E6.
Trapnell, C., Pachter, L., and Salzberg, S.L. (2009). TopHat: discovering splice junctions with RNA-Seq. Bioinformatics 25, 1105-1111.
Trapnell, C., Williams, B.A., Pertea, G., Mortazavi, A., Kwan, G., van Baren, M.J., Salzberg, S.L., Wold, B.J., and Pachter, L. (2010). Transcript assembly and quantification by RNA-Seq reveals unannotated transcripts and isoform switching during cell differentiation. Nature biotechnology 28, 511-515.
Wang, Y., Li, Y., Toth, J.I., Petroski, M.D., Zhang, Z., and Zhao, J.C. (2014). N6-methyladenosine modification destabilizes developmental regulators in embryonic stem cells. Nature cell biology 16, 191-198.
Wei, C.M., Gershowitz, A., and Moss, B. (1975). Methylated nucleotides block 5′ terminus of HeLa cell messenger RNA. Cell 4, 379-386.
Wei, C.M., and Moss, B. (1974). Methylation of newly synthesized viral messenger RNA by an enzyme in vaccinia virus. Proceedings of the National Academy of Sciences of the United States of America 71, 3014-3018.
Wildeboer, D., Naus, S., Amy Sang, Q.X., Bartsch, J.W., and Pagenstecher, A. (2006). Metalloproteinase disintegrins ADAM8 and ADAM19 are highly regulated in human primary brain tumors and their expression levels and activities are associated with invasiveness. Journal of neuropathology and experimental neurology 65, 516-527.
Yue, Y., Liu, J., and He, C. (2015). RNA N6-methyladenosine methylation in post-transcriptional gene expression regulation. Genes & development 29, 1343-1355.
Zhang, C., Samanta, D., Lu, H., Bullen, J.W., Zhang, H., Chen, I., He, X., and Semenza, G.L. (2016). Hypoxia induces the breast cancer stem cell phenotype by HIF-dependent and ALKBH5-mediated m(6)A-demethylation of NANOG mRNA. Proceedings of the National Academy of Sciences of the United States of America 113, E2047-2056.
Zhao, X., Yang, Y., Sun, B.F., Shi, Y., Yang, X., Xiao, W., Hao, Y.J., Ping, X.L., Chen, Y.S., Wang, W.J., et al. (2014). FTO-dependent demethylation of N6-methyladenosine regulates mRNA splicing and is required for adipogenesis. Cell research 24, 1403-1419.
Zhao, Y., Li, J.S., Guo, M.Z., Feng, B.S., and Zhang, J.P. (2010). Inhibitory effect of S-adenosylmethionine on the growth of human gastric cancer cells in vivo and in vitro. Chin J Cancer 29, 752-760.
Zheng, G., Dahl, J.A., Niu, Y., Fedorcsak, P., Huang, C.M., Li, C.J., Vagbo, C.B., Shi, Y., Wang, W.L., Song, S.H., et al. (2013). ALKBH5 is a mammalian RNA demethylase that impacts RNA metabolism and mouse fertility. Molecular cell 49, 18-29.
Zhou, J., Wan, J., Gao, X., Zhang, X., Jaffrey, S.R., and Qian, S.B. (2015). Dynamic m(6)A mRNA methylation directs translational control of heat shock response. Nature 526, 591-594.

1. A method for treating a subject suffering from a cancer comprising contacting a cancer stem cell with an FTO inhibitor, wherein the FTO inhibitor increases the mRNA methylation level in the cancer stem cells of the subject.

2. (canceled)

3. The method of claim 1, wherein the cancer is an RNA methylation related cancer.

4. The method of claim 1, wherein the cancer is glioblastoma, leukemia (e.g., acute myeloid leukemia), stomach cancer, prostate cancer, colorectal cancer, endometrial cancer, breast cancer, pancreatic cancer, kidney cancer, mesothelioma, or sarcoma.

5. (canceled)

6. The method of claim 1, further comprising contacting the cancer stem cell with an ALKBH5 inhibitor, a TLX inhibitor, or a combination thereof.

7. The method of claim 1, wherein the FTO inhibitor is a small molecule FTO inhibitor, an FTO antibody, or an immunogenic fragment thereof, or a small RNA inhibiting FTO.

8. The method of claim 7, wherein the small molecule FTO inhibitor is MA2 or a derivative of MA.

9. The method of claim 7, wherein the small RNA is siRNA, shRNA or miRNA.

10. (canceled)

11. (canceled)

12. The method of claim 1 comprising administering to the subject one or more therapeutic agents that inhibit FTO, that inhibit ALKBH5, or that inhibit TLX.

13. The method of claim 12, wherein the one or more therapeutic agents are administered to the subject simultaneously.

14. The method of claim 12, wherein the one or more therapeutic agents are administered to the subject sequentially.

15. A pharmaceutical composition for treating cancer, comprising one or more therapeutic agents that inhibit FTO.

16. The pharmaceutical composition of claim 15, further comprising a pharmaceutically acceptable carrier.

17. The pharmaceutical composition of claim 15, wherein the pharmaceutical composition is formulated into an injectable formulation or an oral dosage form.

18. The method of claim 1, wherein increasing mRNA methylation level in the cancer stem cells reduces cancer stem cell growth.

19. The method of claim 1, wherein increasing mRNA methylation level in the cancer stem cells inhibits cancer stem cell self-renewal.

20. The method of claim 1, wherein the subject experiences reduced tumor size.

21. The method of claim 1, wherein the subject experiences increased survival.

22. The method of claim 1, wherein the FTO inhibitor is administered intracranially or intravenously.


SEQUENCE LISTING (21 sequences)

SEQ ID NO: 1 - DNA, 21 bp, synthetic construct
  misc_feature 1..21: note = control shRNA
  source 1..21: mol_type = other DNA
actcaaaaggaagtgacaaga

SEQ ID NO: 2 - DNA, 19 bp, synthetic construct
  misc_feature 1..19: note = METTL3 shRNA-1
  source 1..19: mol_type = other DNA
gctgcacttcagacgaatt

SEQ ID NO: 3 - DNA, 19 bp, synthetic construct
  misc_feature 1..19: note = METTL3 shRNA-2
  source 1..19: mol_type = other DNA
ccacctcagtggatctgtt

SEQ ID NO: 4 - DNA, 18 bp, synthetic construct
  misc_feature 1..18: note = METTL14 shRNA-1
  source 1..18: mol_type = other DNA
gctaaaggatgagttaat

SEQ ID NO: 5 - DNA, 19 bp, synthetic construct
  misc_feature 1..19: note = METTL14 shRNA-2
  source 1..19: mol_type = other DNA
ggacttgggatgatattat

SEQ ID NO: 6 - DNA, 22 bp, synthetic construct
  misc_feature 1..22: note = ADAM19 shRNA-1
  source 1..22: mol_type = other DNA
ggaagatttaaactccatgaag

SEQ ID NO: 7 - DNA, 22 bp, synthetic construct
  misc_feature 1..22: note = ADAM19 shRNA-2
  source 1..22: mol_type = other DNA
caaagtgttcaatggatgcaac

SEQ ID NO: 8 - DNA, 21 bp, synthetic construct
  misc_feature 1..21: note = METLL3 forward primer
  source 1..21: mol_type = other DNA
tcagcatcggaaccagcaaag

SEQ ID NO: 9 - DNA, 21 bp, synthetic construct
  misc_feature 1..21: note = METLL3 reverse primer
  source 1..21: mol_type = other DNA
tcctgactgaccttcttgctc

SEQ ID NO: 10 - DNA, 20 bp, synthetic construct
  misc_feature 1..20: note = METLL14 forward primer
  source 1..20: mol_type = other DNA
gttggaacatggatagccgc

SEQ ID NO: 11 - DNA, 20 bp, synthetic construct
  misc_feature 1..20: note = METLL14 reverse primer
  source 1..20: mol_type = other DNA
caatgctgtcggcactttca

SEQ ID NO: 12 - DNA, 20 bp, synthetic construct
  misc_feature 1..20: note = CD44 forward primer
  source 1..20: mol_type = other DNA
tgagcatcggatttgagacc

SEQ ID NO: 13 - DNA, 21 bp, synthetic construct
  misc_feature 1..21: note = CD44 reverse primer
  source 1..21: mol_type = other DNA
tgtcatactgggaggtgttgg

SEQ ID NO: 14 - DNA, 20 bp, synthetic construct
  misc_feature 1..20: note = ADAM19 forward primer
  source 1..20: mol_type = other DNA
cctggatggacaagaggaag

SEQ ID NO: 15 - DNA, 20 bp, synthetic construct
  misc_feature 1..20: note = ADAM19 reverse primer
  source 1..20: mol_type = other DNA
ctcagctttgagtggatgct

SEQ ID NO: 16 - DNA, 21 bp, synthetic construct
  misc_feature 1..21: note = EPAH3 forward primer
  source 1..21: mol_type = other DNA
gggctggatctcttatccatc

SEQ ID NO: 17 - DNA, 20 bp, synthetic construct
  misc_feature 1..20: note = EPAH3 reverse primer
  source 1..20: mol_type = other DNA
ggacccagtttgttctcagc

SEQ ID NO: 18 - DNA, 20 bp, synthetic construct
  misc_feature 1..20: note = KLF4 forward primer
  source 1..20: mol_type = other DNA
aagagttcccatctcaaggc

SEQ ID NO: 19 - DNA, 20 bp, synthetic construct
  misc_feature 1..20: note = KLF4 reverse primer
  source 1..20: mol_type = other DNA
ccgtgtgtttacggtagtgc

SEQ ID NO: 20 - DNA, 22 bp, synthetic construct
  misc_feature 1..22: note = ACTIN forward primer
  source 1..22: mol_type = other DNA
ccgcaaagacctgtacgccaac

SEQ ID NO: 21 - DNA, 22 bp, synthetic construct
  misc_feature 1..22: note = ACTIN reverse primer
  source 1..22: mol_type = other DNA
ccagggcagtgatctccttctg